SEQUENCE LISTING (72 sequences)

SEQ ID NO: 1 - DNA, 20 bp, Herpes Simplex Virus-1
ataccgaccacaccgacgaa

SEQ ID NO: 2 - DNA, 23 bp, Herpes Simplex Virus-1
acgcaccacacaaaagagacctt

SEQ ID NO: 3 - DNA, 23 bp, Herpes Simplex Virus-1
cttcagcgcgaacgaccaactac

SEQ ID NO: 4 - DNA, 19 bp, Herpes Simplex Virus-2
cgccaaatacgccttagca

SEQ ID NO: 5 - DNA, 20 bp, Herpes Simplex Virus-2
gaaggttcttcccgcgaaat

SEQ ID NO: 6 - DNA, 20 bp, Herpes Simplex Virus-2
cccctcgcttaagatggccg

SEQ ID NO: 7 - DNA, 18 bp, Herpes Simplex Virus-1
ataccgaccacaccgacg

SEQ ID NO: 8 - DNA, 18 bp, Herpes Simplex Virus-1
accgaccacaccgacgaa

SEQ ID NO: 9 - DNA, 16 bp, Herpes Simplex Virus-1
accgaccacaccgacg

SEQ ID NO: 10 - DNA, 18 bp, Herpes Simplex Virus-1
taccgaccacaccgacga

SEQ ID NO: 11 - DNA, 19 bp, Herpes Simplex Virus-1
taccgaccacaccgacgaa

SEQ ID NO: 12 - DNA, 19 bp, Herpes Simplex Virus-1
ataccgaccacaccgacga

SEQ ID NO: 13 - DNA, 17 bp, Herpes Simplex Virus-1
accgaccacaccgacga

SEQ ID NO: 14 - DNA, 17 bp, Herpes Simplex Virus-1
taccgaccacaccgacg

SEQ ID NO: 15 - DNA, 22 bp, Herpes Simplex Virus-1
ataccgaccacaccgacgaatc

SEQ ID NO: 16 - DNA, 22 bp, Herpes Simplex Virus-1
ccataccgaccacaccgacgaa

SEQ ID NO: 17 - DNA, 26 bp, Herpes Simplex Virus-1
gaattcataccgaccacaccgacgaa

SEQ ID NO: 18 - DNA, 21 bp, Herpes Simplex Virus-1
acgcaccacacaaaagagacc

SEQ ID NO: 19 - DNA, 21 bp, Herpes Simplex Virus-1
gcaccacacaaaagagacctt

SEQ ID NO: 20 - DNA, 19 bp, Herpes Simplex Virus-1
gcaccacacaaaagagacc

SEQ ID NO: 21 - DNA, 21 bp, Herpes Simplex Virus-1
cgcaccacacaaaagagacct

SEQ ID NO: 22 - DNA, 22 bp, Herpes Simplex Virus-1
cgcaccacacaaaagagacctt

SEQ ID NO: 23 - DNA, 22 bp, Herpes Simplex Virus-1
acgcaccacacaaaagagacct

SEQ ID NO: 24 - DNA, 20 bp, Herpes Simplex Virus-1
gcaccacacaaaagagacct

SEQ ID NO: 25 - DNA, 20 bp, Herpes Simplex Virus-1
cgcaccacacaaaagagacc

SEQ ID NO: 26 - DNA, 25 bp, Herpes Simplex Virus-1
acgcaccacacaaaagagaccttaa

SEQ ID NO: 27 - DNA, 25 bp, Herpes Simplex Virus-1
gaacgcaccacacaaaagagacctt

SEQ ID NO: 28 - DNA, 29 bp, Herpes Simplex Virus-1
gaattcacgcaccacacaaaagagacctt

SEQ ID NO: 29 - DNA, 21 bp, Herpes Simplex Virus-1
cttcagcgcgaacgaccaact

SEQ ID NO: 30 - DNA, 21 bp, Herpes Simplex Virus-1
tcagcgcgaacgaccaactac

SEQ ID NO: 31 - DNA, 19 bp, Herpes Simplex Virus-1
tcagcgcgaacgaccaact

SEQ ID NO: 32 - DNA, 21 bp, Herpes Simplex Virus-1
ttcagcgcgaacgaccaacta

SEQ ID NO: 33 - DNA, 22 bp, Herpes Simplex Virus-1
ttcagcgcgaacgaccaactac

SEQ ID NO: 34 - DNA, 22 bp, Herpes Simplex Virus-1
cttcagcgcgaacgaccaacta

SEQ ID NO: 35 - DNA, 20 bp, Herpes Simplex Virus-1
tcagcgcgaacgaccaacta

SEQ ID NO: 36 - DNA, 20 bp, Herpes Simplex Virus-1
ttcagcgcgaacgaccaact

SEQ ID NO: 37 - DNA, 25 bp, Herpes Simplex Virus-1
cttcagcgcgaacgaccaactaccc

SEQ ID NO: 38 - DNA, 25 bp, Herpes Simplex Virus-1
agcttcagcgcgaacgaccaactac

SEQ ID NO: 39 - DNA, 17 bp, Herpes Simplex Virus-2
cgccaaatacgccttag

SEQ ID NO: 40 - DNA, 17 bp, Herpes Simplex Virus-2
ccaaatacgccttagca

SEQ ID NO: 41 - DNA, 15 bp, Herpes Simplex Virus-2
ccaaatacgccttag

SEQ ID NO: 42 - DNA, 17 bp, Herpes Simplex Virus-2
gccaaatacgccttagc

SEQ ID NO: 43 - DNA, 18 bp, Herpes Simplex Virus-2
gccaaatacgccttagca

SEQ ID NO: 44 - DNA, 18 bp, Herpes Simplex Virus-2
cgccaaatacgccttagc

SEQ ID NO: 45 - DNA, 16 bp, Herpes Simplex Virus-2
ccaaatacgccttagc

SEQ ID NO: 46 - DNA, 16 bp, Herpes Simplex Virus-2
gccaaatacgccttag

SEQ ID NO: 47 - DNA, 21 bp, Herpes Simplex Virus-2
cgccaaatacgccttagcaga

SEQ ID NO: 48 - DNA, 21 bp, Herpes Simplex Virus-2
tgcgccaaatacgccttagca

SEQ ID NO: 49 - DNA, 25 bp, Herpes Simplex Virus-2
gaattccgccaaatacgccttagca

SEQ ID NO: 50 - DNA, 18 bp, Herpes Simplex Virus-2
gaaggttcttcccgcgaa

SEQ ID NO: 51 - DNA, 18 bp, Herpes Simplex Virus-2
aggttcttcccgcgaaat

SEQ ID NO: 52 - DNA, 16 bp, Herpes Simplex Virus-2
aggttcttcccgcgaa

SEQ ID NO: 53 - DNA, 18 bp, Herpes Simplex Virus-2
aaggttcttcccgcgaaa

SEQ ID NO: 54 - DNA, 19 bp, Herpes Simplex Virus-2
aaggttcttcccgcgaaat

SEQ ID NO: 55 - DNA, 19 bp, Herpes Simplex Virus-2
gaaggttcttcccgcgaaa

SEQ ID NO: 56 - DNA, 17 bp, Herpes Simplex Virus-2
aggttcttcccgcgaaa

SEQ ID NO: 57 - DNA, 17 bp, Herpes Simplex Virus-2
aaggttcttcccgcgaa

SEQ ID NO: 58 - DNA, 22 bp, Herpes Simplex Virus-2
gaaggttcttcccgcgaaatcg

SEQ ID NO: 59 - DNA, 22 bp, Herpes Simplex Virus-2
cggaaggttcttcccgcgaaat

SEQ ID NO: 60 - DNA, 26 bp, Herpes Simplex Virus-2
gaattcgaaggttcttcccgcgaaat

SEQ ID NO: 61 - DNA, 18 bp, Herpes Simplex Virus-2
cccctcgcttaagatggc

SEQ ID NO: 62 - DNA, 18 bp, Herpes Simplex Virus-2
cctcgcttaagatggccg

SEQ ID NO: 63 - DNA, 16 bp, Herpes Simplex Virus-2
cctcgcttaagatggc

SEQ ID NO: 64 - DNA, 18 bp, Herpes Simplex Virus-2
ccctcgcttaagatggcc

SEQ ID NO: 65 - DNA, 19 bp, Herpes Simplex Virus-2
ccctcgcttaagatggccg

SEQ ID NO: 66 - DNA, 19 bp, Herpes Simplex Virus-2
cccctcgcttaagatggcc

SEQ ID NO: 67 - DNA, 17 bp, Herpes Simplex Virus-2
cctcgcttaagatggcc

SEQ ID NO: 68 - DNA, 17 bp, Herpes Simplex Virus-2
ccctcgcttaagatggc

SEQ ID NO: 69 - DNA, 22 bp, Herpes Simplex Virus-2
cccctcgcttaagatggccgat

SEQ ID NO: 70 - DNA, 22 bp, Herpes Simplex Virus-2
gacccctcgcttaagatggccg

SEQ ID NO: 71 - DNA, 21 bp, Herpes Simplex Virus-1
ccataccgaccacaccgacga

SEQ ID NO: 72 - DNA, 25 bp, Herpes Simplex Virus-2
agcgcgaacgaccaactaccccgat

(19) United States
(12) Patent Application Publication
(10) Pub. No.: US 2006/0141481 A1
(43) Pub. Date: Jun. 29, 2006

(54) HSV-1 and HSV-2 primers and probes

(76) Inventor: Brian D. Mariani, Herndon, VA (US)

Correspondence Address: MCDERMOTT WILL & EMERY LLP, 600 13TH STREET, N.W., WASHINGTON, DC, 20005-3096, US

(21) Appl. No.: 11/020,676

(22) Filed: Dec. 27, 2004

(51) Int. Cl. C12Q 1/70; C12Q 1/68

(52) U.S. Cl. 435/6; 435/5

(57) ABSTRACT

The present invention provides methods, primers and probes for the detection of HSV nucleic acids in biological fluids and tissue. In the methods of the invention, at least a portion of HSV nucleic acid present in a biological sample suspected of containing an HSV-1 and/or HSV-2 is amplified and the amplified HSV nucleic acid is then detected. Detection may be accomplished by conventional separation techniques such as gel electrophoresis or by hybridization of at least a portion of a nucleotide probe comprising a nucleotide sequence complementary to the amplified HSV nucleic acid. Preferably, HSV DNA is detected in a biological sample using real-time PCR techniques that can detect the increasing presence of an amplification product while amplification occurs.

### BACKGROUND OF THE INVENTION

1. Field of the Invention

The present invention relates to primers, probes and methods for detecting viral infection of biological tissues, and more particularly to primers, probes and methods for detecting HSV-1 and/or HSV-2 infection of biological fluids and tissues using the primers and probes.

2. Background

The ongoing revolution in genomic sciences has provided the biomedical community with unique opportunities to advance medical diagnosis using molecular methods. This wealth of new genetic information is being utilized by the diagnostic community to develop molecular-based assays for pathogen detection to expand detection capabilities. The complete genomes for over 1,000 microorganisms (viral, bacterial, parasitic) have been compiled and made available as public databases (http://www.tigr.org), with many more strain and subtypes variants being added almost daily (http://www.ncbi.nlm.nih.gov).

Molecular diagnostics in the clinic is now possible due to the ability to amplify small amounts of genetic material in a patient specimen using such methods as the polymerase chain reaction (PCR), ligase chain reaction (LCR), or transcription mediated amplification (TMA), as well as others. While many of these technologies have been incorporated into automated commercial systems, PCR holds the greatest flexibility for user-developed methods when the concern is addressing clinical needs not covered by fully automated platforms. This is especially true for neonatal Herpes Simplex Virus (HSV) testing where accuracy and time-to-results are crucial for establishing proper treatment regimens, improved patient outcomes, and rational hospital resource allocation.

Herpes Simplex Virus (HSV) infection elicits a wide range of clinical symptoms in both infants and adults and is the cause of 10-20% of viral encephalitis in the United States. It circulates as two related but distinct serotypes, type 1 and type 2. Herpes simplex virus type 1 (HSV-1) infection usually involves areas of the lips, mouth, nostrils, and face. HSV-1 is the most common serotype in the general population and is usually contracted during childhood.

HSV-1 lesions of the mouth region, known as cold sores or fever blisters allow transmission by contact with infected saliva. However, viral shedding is common before the appearance of lesions and transmitted in the absense of symptoms. In the U.S., at least 90% of adults will exhibit antibodies to HSV-1.

Herpes simplex virus 2 (HSV-2) is usually transmitted via sexual activity and is associated with genital ulcers or sores during primary (initial) infection. Individuals may “carry” HSV-2 in a latent state without ever developing symptoms. Viral transmission may occur however even in asymptomatic carriers. Approximately 30-40% of adults in the U.S. have antibodies against HSV-2.

HSV infection can result in complications of the central nervous system, most typically, meningoencephalitis (infection of the lining of the brain and the brain itself), or infection of the eye, particularly the conjunctiva, and the cornea. Herpetic whitlow is a form of HSV infection involving the finger, and is found affecting health care workers (because of exposure to oral secretions) and young children. Significantly, HSV is also a problem during pregnancy and may infect the fetus through the maternal blood supply and cause congenital abnormalities.

HSV transmission to the newborn during vaginal delivery in mothers infected with the virus (with or without lesions) is of great concern the medical community. Transmission is more likely if the mother has an active infection at delivery, indicating maternal primary infection. In this case, the mother has not yet mounted an immune response to attenuate viral replication, nor can she transfer maternal immunity on to the newborn. Undiagnosed neonatal HSV infection can have dire consequences for the infant, with long-term neurological impairment, or infant mortality.

During the normal life cycle of HSV it spreads to nerve cells underlying the site of contact and remains dormant. It may periodically reactivate in response to the host's immunological state and cause recurrent symptoms or flares. Reactivation may be initiated by overexposure to sunlight, fever, stress, acute illness, or other conditions that weaken the immune system. Reactivation and the associated patient morbidity typically occurs during cancer, HIV/AIDS, organ transplant recipients, and/or use of corticosteroid medications.

The diagnosis of neonatal HSV can be difficult, and it should be suspected in any newborn with irritability, lethargy, fever or poor feeding at one week of age. For the neonate, molecular methods serve as an augmentation to and not a displacement of existing assays. Traditional bacteriology is still the method of choice for the suspicion of sepsis, when outgrowth and identification of bacterial infection can be achieved at low cost in 1-2 days, and during which time antibiotic treatment can be started prior to results, when appropriate. The situation is more complicated for the suspicion of viral infection however, since traditional viral culture or immunofluorescent methods can be lengthier and false-negative rates a reality. Serological testing of the newborn is most often uninformative due to the presence of maternal antibodies, unless the mother is accessed independently in order to determine, for example, if a primary verse secondary (or reactivated) infection is evident, where the former case would be more problematic for the newborn, compared to the latter.

The value of the PCR in the acute phase diagnosis of HSV infection lies in its ability to detect nucleic acid at very low levels. Application of herpes simplex virus polymerase chain reaction detection to CSF has been shown to be a sensitive and specific test. See Lakeman et al. J Infect Dis. April 1995; 171(4):857-63.

However, the use of DNA amplification techniques can be problematic. The procedures are susceptible to false-positive or false-negative results therefore, proper picking of primers and probes as well as careful handling of the test sample, are essential. Landry (J Infect Dis 1995; 172:1641-3).

The instant invention provides for a specific PCR assay to determine the presence of HSV-1 and/or HSV-2 DNA as well as differentiate accurately and definitively between the presence of HSV-1 and HSV-2 DNA.

All publications, scientific, patent or otherwise are hereby incorporated by reference in their entirety for all purposes.

### SUMMARY OF THE INVENTION

One aspect of the invention relates to an isolated oligonucleotide of the sequence SEQ ID NO: 1. One embodiment of this aspect of the invention relates to an isolated oligonucleotide that hybridizes the complement of SEQ ID NO: 1 under stringent conditions and is capable of amplifying an HSV-1 glycoprotein D gene when used in conjunction with SEQ ID NO: 2 in an polymerase chain reaction. Another embodiment of this aspect of the invention relates to an isolated oligonucleotide of the sequence of SEQ ID NO: 1, wherein about one to about three nucleotides are added or removed from the 5′ end and/or about one to about three nucleotides are added or removed from the 3′ end, respectively.

Another aspect of the invention relates to an isolated oligonucleotide of the sequence SEQ ID NO: 2. One embodiment of this aspect of the invention relates to an isolated oligonucleotide that hybridizes the complement of SEQ ID NO: 2 under stringent conditions and is capable of amplifying the HSV-1 glycoprotein D gene when used in conjunction with SEQ ID NO: 1 in an polymerase chain reaction. Another embodiment relates to an isolated oligonucleotide of the sequence of SEQ ID NO: 2, wherein about one to about three nucleotides are added or removed from the 5′ end and/or about one to about three nucleotides are added or removed from the 3′ end, respectively.

Another aspect of the invention relates to an isolated oligonucleotide having the sequence of SEQ ID NO: 3 or a sequence wherein about one to about three nucleotides are added or removed from the 5′ end and/or about one to about three nucleotides are added or removed from the 3′ end of SEQ ID NO: 3.

Another aspect of the invention relates to an isolated oligonucleotide of the sequence SEQ ID NO: 4. One embodiment of this aspect of the invention relates to an isolated oligonucleotide that hybridizes the complement of SEQ ID NO: 4 under stringent conditions and is capable of amplifying the HSV-2 glycoprotein G gene when used in conjunction with SEQ ID NO: 5 in an polymerase chain reaction. Another embodiment of this aspect of the invention relates to an isolated oligonucleotide of the sequence of SEQ ID NO: 4, wherein about one to about three nucleotides are added or removed from the 5′ end and/or about one to about three nucleotides are added or removed from the 3′ end, respectively.

Another aspect of the invention relates to an isolated oligonucleotide of the sequence SEQ ID NO: 5. One embodiment of this aspect of the invention relates to an isolated oligonucleotide that hybridizes the complement of SEQ ID NO: 5 under stringent conditions and is capable of amplifying the HSV-2 glycoprotein G gene when used in conjunction with SEQ ID NO: 4 in an polymerase chain reaction. Another embodiment relates to an isolated oligonucleotide of the sequence of SEQ ID NO: 5, wherein about one to about three nucleotides are added or removed from the 5′ end and/or about one to about three nucleotides are added or removed from the 3′ end, respectively.

Another aspect of the invention relates to an isolated oligonucleotide having the sequence of SEQ ID NO: 6 or a sequence wherein about one to about three nucleotides are added or removed from the 5′ end and/or about one to about three nucleotides are added or removed from the 3′ end of SEQ ID NO: 6.

Another aspect of the invention relates to a kit for detecting a HSV-1 glycoprotein D gene comprising a first isolated oligonucleotide of SEQ ID NO: 1 and a second oligonucleotide of SEQ ID NO: 2 or an oligonucleotide substantially identical thereto.

Another aspect of the invention relates to a kit for detecting a HSV-1 glycoprotein D gene comprising a first isolated oligonucleotide of SEQ ID NO: 2 and a second oligonucleotide of SEQ ID NO: 1 or an oligonucleotide substantially identical thereto.

Another aspect of the invention relates to a kit for detecting a HSV-1 glycoprotein D gene comprising a first isolated oligonucleotide of SEQ ID NO: 1 and a second oligonucleotide of SEQ ID NO: 2.

Another aspect of the invention relates to a kit for detecting a HSV-1 glycoprotein D gene comprising a first oligonucleotide selected from the group consisting of: an isolated oligonucleotide of the sequence SEQ ID NO: 1; an isolated oligonucleotide that hybridizes the complement of SEQ ID NO: 1 under stringent conditions and is capable of amplifying the HSV-1 glycoprotein D gene when used in conjunction with SEQ ID NO: 2 in an polymerase chain reaction; and an isolated oligonucleotide of the sequence of SEQ ID NO: 1, wherein about one to about three nucleotides are added or removed from the 5′ end and/or about one to about three nucleotides are added or removed from the 3′ end, respectively; and a second oligonucleotide selected from the group consisting of an isolated oligonucleotide of the sequence SEQ ID NO: 2; an isolated oligonucleotide that hybridizes the complement of SEQ ID NO: 2 under stringent conditions and is capable of amplifying the HSV-1 glycoprotein D gene when used in conjunction with SEQ ID NO: 1 in an polymerase chain reaction; and an isolated oligonucleotide of the sequence of SEQ ID NO: 2, wherein about one to about three nucleotides are added or removed from the 5′ end and/or about one to about three nucleotides are added or removed from the 3′ end, respectively.

Another aspect of the invention relates to a method of detecting the presence of a HSV-1 glycoprotein D gene in a biological sample comprising: obtaining a biological sample from an organism; isolating nucleic acids from the sample; performing a polymerase chain reaction on the isolated nucleic acids using a first isolated oligonucleotide selected from the group consisting of: an isolated oligonucleotide of the sequence SEQ ID NO: 1; an isolated oligonucleotide that hybridizes the complement of SEQ ID NO: 1 under stringent conditions and is capable of amplifying the HSV-1 glycoprotein D gene when used in conjunction with SEQ ID NO: 2 in an polymerase chain reaction; and an isolated oligonucleotide of the sequence of SEQ ID NO: 1, wherein about one to about three nucleotides are added or removed from the 5′ end and/or about one to about three nucleotides are added or removed from the 3′ end, respectively; and a second oligonucleotide selected from the group consisting of an isolated oligonucleotide of the sequence SEQ ID NO: 2; an isolated oligonucleotide that hybridizes the complement of SEQ ID NO: 2 under stringent conditions and is capable of amplifying the HSV-1 glycoprotein D gene when used in conjunction with SEQ ID NO: 1 in an polymerase chain reaction; and an isolated oligonucleotide of the sequence of SEQ ID NO: 2, wherein about one to about three nucleotides are added or removed from the 5′ end and/or about one to about three nucleotides are added or removed from the 3′ end, respectively, correlating a presence of an amplification product from the polymerase chain reaction with the presence of the HSV-1 glycoprotein D gene is the sample.

Another aspect of the invention relates to a kit for detecting a HSV-2 glycoprotein G gene comprising a first isolated oligonucleotide of SEQ ID NO: 4 and a second oligonucleotide of SEQ ID NO: 5 or an oligonucleotide substantially identical thereto.

Another aspect of the invention relates to a kit for detecting a HSV-2 glycoprotein G gene comprising a first isolated oligonucleotide of SEQ ID NO: 5 and a second oligonucleotide of SEQ ID NO: 4 or an oligonucleotide substantially identical thereto.

Another aspect of the invention relates to a kit for detecting a HSV-2 glycoprotein G gene comprising a first isolated oligonucleotide of SEQ ID NO: 4 and a second oligonucleotide of SEQ ID NO: 5.

Another aspect of the invention relates to a kit for detecting a HSV-2 glycoprotein G gene comprising a first oligonucleotide selected from the group consisting of: an isolated oligonucleotide of the sequence SEQ ID NO: 4; an isolated oligonucleotide that hybridizes the complement of SEQ ID NO: 4 under stringent conditions and is capable of amplifying the HSV-2 glycoprotein G gene when used in conjunction with SEQ ID NO: 5 in an polymerase chain reaction; and an isolated oligonucleotide of the sequence of SEQ ID NO: 4, wherein about one to about three nucleotides are added or removed from the 5′ end and/or about one to about three nucleotides are added or removed from the 3′ end, respectively; and a second oligonucleotide selected from the group consisting of an isolated oligonucleotide of the sequence SEQ ID NO: 5; an isolated oligonucleotide that hybridizes the complement of SEQ ID NO: 5 under stringent conditions and is capable of amplifying the HSV-2 glycoprotein G gene when used in conjunction with SEQ ID NO: 4 in an polymerase chain reaction; and an isolated oligonucleotide of the sequence of SEQ ID NO: 5, wherein about one to about three nucleotides are added or removed from the 5′ end and/or about one to about three nucleotides are added or removed from the 3′ end, respectively.

Another aspect of the invention relates to a method of detecting the presence of a HSV-2 glycoprotein G gene in a biological sample comprising: obtaining a biological sample from an organism; isolating nucleic acids from the sample; performing a polymerase chain reaction on the isolated nucleic acids using a first isolated oligonucleotide selected from the group consisting of: an isolated oligonucleotide of the sequence SEQ ID NO: 4; an isolated oligonucleotide that hybridizes the complement of SEQ ID NO: 4 under stringent conditions and is capable of amplifying the HSV-2 glycoprotein G gene when used in conjunction with SEQ ID NO: 5 in an polymerase chain reaction; and an isolated oligonucleotide of the sequence of SEQ ID NO: 4, wherein about one to about three nucleotides are added or removed from the 5′ end and/or about one to about three nucleotides are added or removed from the 3′ end, respectively; and a second oligonucleotide selected from the group consisting of an isolated oligonucleotide of the sequence SEQ ID NO: 5; an isolated oligonucleotide that hybridizes the complement of SEQ ID NO: 5 under stringent conditions and is capable of amplifying the HSV-2 glycoprotein G gene when used in conjunction with SEQ ID NO: 4 in an polymerase chain reaction; and an isolated oligonucleotide of the sequence of SEQ ID NO: 5, wherein about one to about three nucleotides are added or removed from the 5′ end and/or about one to about three nucleotides are added or removed from the 3′ end, respectively, correlating a presence of an amplification product from the polymerase chain reaction with the presence of the HSV-2 glycoprotein G gene is the sample.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. The present invention may be practiced without some or all of these specific details. In other instances, well known process operations have not been described in detail, in order not to unnecessarily obscure the present invention. Accordingly, the drawings and description are to be regarded as illustrative in nature, and not as restrictive.

### BRIEF DESCRIPTION OF THE DRAWINGS

FIG. 1. Real-time PCR using hydrolysis probes. During the DNA amplification process, an amplicon-specific oligonucleotide probe hybridizes to one strand of the amplicon. The probe contains a 5′-reporter fluorochrome (blue) and a 3′-quencher fluorochrome (green) Fluorescence emission from the reporter is absorbed (quenched) by the quencher on the intact probe due to the close proximity of the 2 moieties. As the target amplicon is generated, the probe binds to the complementary amplicon single strand during the annealing phase of the PCR cycle. The DNA polymerase extending from the downstream primer hydrolyzes the probe by virtue of its 5′ exonucleotide activity cleaving the reporter from the probe. Once released physically from the quencher, the reporter can fluoresce allowing detection by the instrument.

FIG. 2. Extraction flowchart: Nucleic acid binding column method results in rapid purification of PCR-ready RNA/DNA from a variety of clinical sources.

FIG. 3. HSV envelope: HSV glycoproteins are clearly visible in clusters of spikes of approximately 10 nm length. Between the capsid and the envelope is an ill-defined layer of proteins, collectively known as the tegument. Image Courtesy of Linda Stannard, of the Department of Medical Microbiology, University of Cape Town

FIG. 4. HSV real-time plots: Plot A shows a dilution series of a mixture HSV-1 and HSV-2 genomic DNA, 10 (blue), 100 (pale blue), 1,000, (orange), 10,000 (green) genomes/assay. This graph shows the data for HSV-1 specific fluorescence (FAM).

FIG. 5. HSV real-time plots: Plot A shows a dilution series of a mixture HSV-1 and HSV-2 genomic DNA, 10 (blue), 100 (pale blue), 1,000, (orange), 10,000 (green) genomes/assay. This graph shows the data for HSV-2 specific fluorescence (ROX).

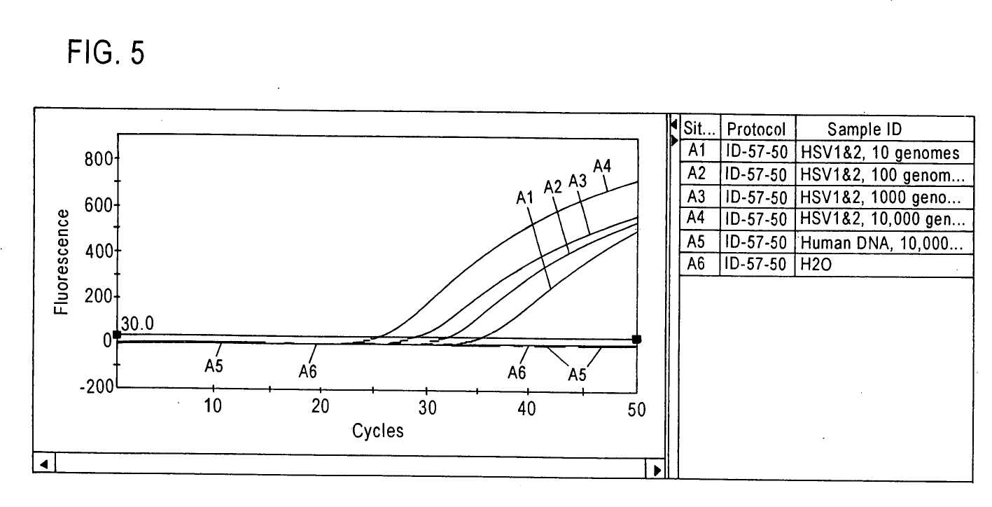

FIG. 6. Internal positive control: Plot shows the detection of patient-derived DNA (TAMRA) in from a variety of HSV-negative specimen types including fractionated blood, CSF, swabs, urine, and amniotic fluid (listed on right).

FIG. 7. Detection of HSV in a clinical specimen. The plot shows the data from 3 clinical specimens (tested in duplicate). Reactions A 12 (light green) and A 13 (brown) show the detection of HSV-2 in neonatal cerebral spinal fluid (ROX). The other 2 specimens are negative for HSV. The HSV positive control (A14) is shown in red.

FIG. 8. Detection of HSV in a clinical specimen. The plot shows the data from 2 clinical specimens (tested in duplicate). Reactions A9 (blue) and A 10 (light blue) show the detection of HSV-1 in neonatal cerebral spinal fluid (ROX). The other specimen is negative for HSV. The HSV positive control (A13) is shown in light green).

FIG. 9. Detection of HSV-1 in confirmed clinical specimens. The plot shows the detection of HSV-1 (FAM) in cerebral spinal fluid from 5 patients with confirmed HSV infection (Series 14204, A1-A5).

FIG. 10. Detection of HSV-2 in confirmed clinical specimens. The plot shows the detection of HSV-2 (ROX) in cerebral spinal fluid from 5 patients with clinically confirmed HSV infection (Series 14232, A6-A10).

### DETAILED DESCRIPTION OF THE INVENTION

The application of a method coupling DNA amplification and product detection has moved molecular diagnostics to a new level of sophistication and utility, especially when speed and accuracy are required. For neonatal testing, high priority is placed on the detection of both HSV-1 and HSV-2, since undetected or untreated HSV infection poses serious health risks for the newborn, normally presenting in one of 3 forms of disease ranging from infection of 1) the skin, eye, and mouth (SEM), 2) the central nervous system (CNS), and/or 3) multiple internal organs in disseminated infection, with or without CNS involvement. Generally, neonatal CNS and/or disseminated infection is associated with severe patient morbidity, even mortality, if not properly treated. In disseminated infection, the herpes virus can affect many different internal organs including the liver, lungs, kidneys, and brain. Likewise undiagnosed and untreated SEM disease can progress to these more serious forms of infection and associated sequela.

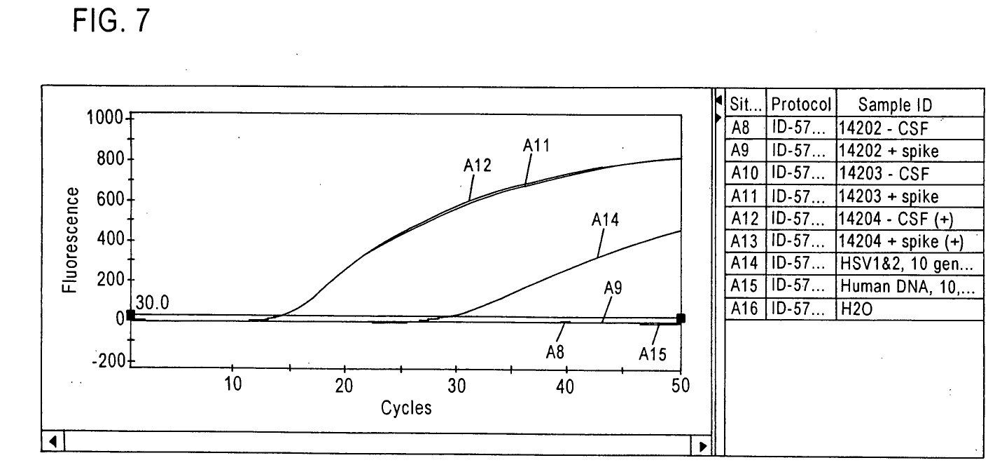

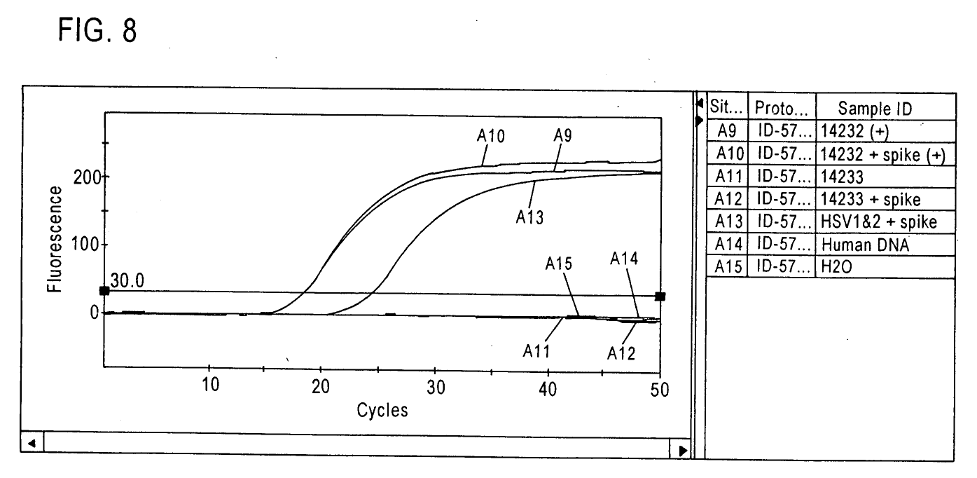

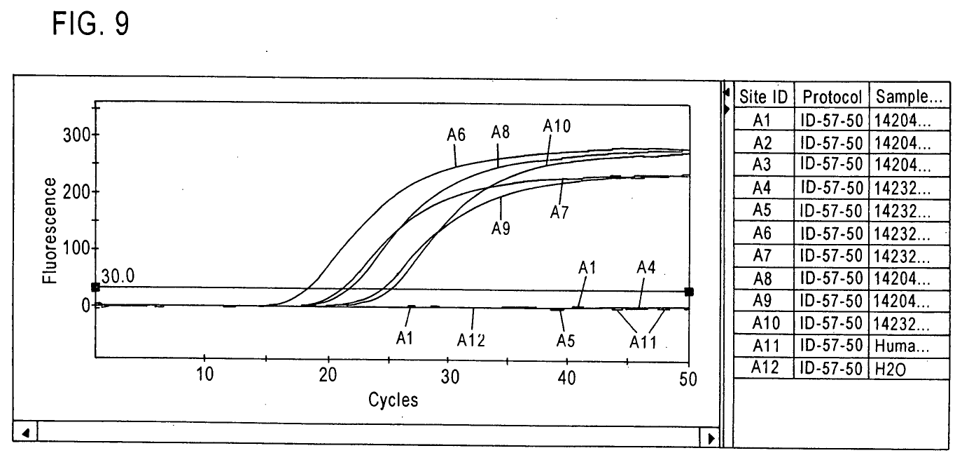

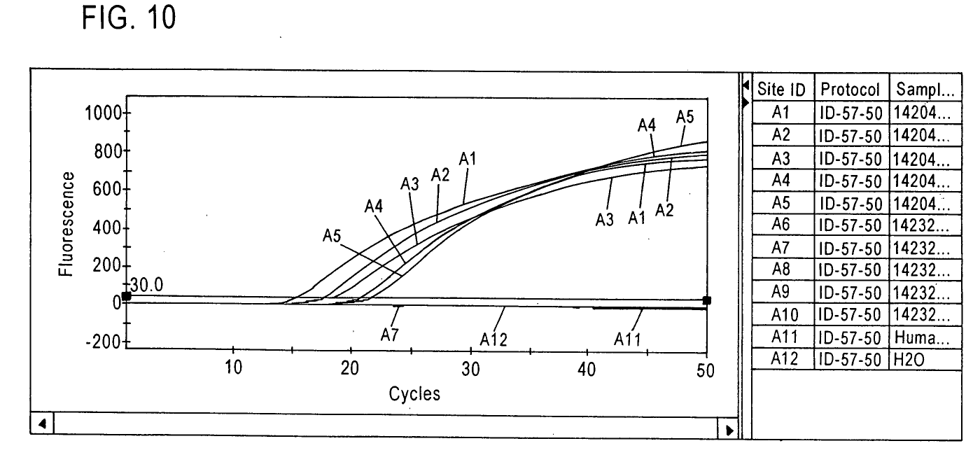

Testing for HSV in cases of neonatal fever, irritability, seizure, lethargy, etc., or in cases where the mother presents symptoms consistent with HSV infection, before, during or after birth, with a rapid, accurate assay provides greater diagnostic confidence that can significantly improve patient management. In the case of a definitive HSV-negative result, the neonate will spend less (or no) time in the NICU, consume less (if any) anti-viral medication, and have a shorter time to discharge, with less additional, potentially expensive diagnostic procedures. Conversely, with rapid time-to-results, the HSV-positive newborn (e.g., FIGS. 7-10) can be treated promptly and correctly, increasing the chances of effective therapy and improving clinical outcome for this dangerous viral infection.

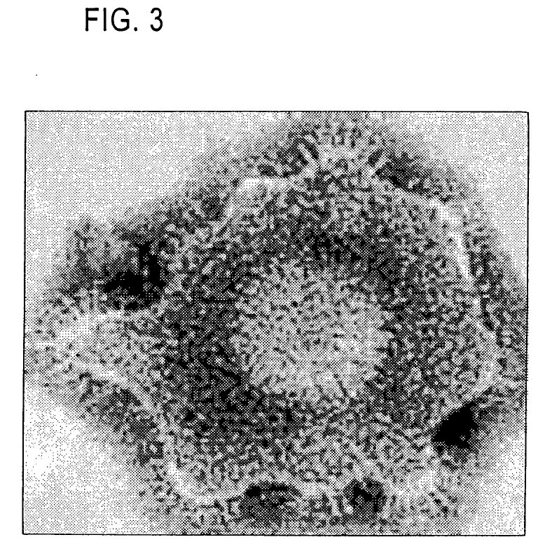

One challenge for HSV testing is detecting both type 1 and type 2 serotypes with equal sensitivity, while distinguishing between serotypes in the same assay. Since both serotypes harbor highly conserved, large˜160,000 basepair (bp) DNA genomes in which many of their˜100 gene sequences are nearly identical, some knowledge of the tropism of HSV infection can guide assay design. Since HSV type 1 and type 2 have tropism for oral and urogenital mucosa, respectively, their respective cell attachment proteins may have structural differences conferring this host cell specificity. Consistent with this idea, it is the case that several of these envelope glycoproteins (FIG. 3) shows type-specific sequence differences that provide appropriate genetic targets that allow type 1 and type 2 differentiation.

The present invention provides methods, primers and probes for the detection of HSV-1 or HSV-2 infections in biological fluids and tissues. In the methods of the invention, at least a portion of HSV nucleic acid present in a biological sample suspected of containing an HSV-1 or HSV-2 is amplified (i.e. multiple copies of the nucleic acid are made) and the amplified nucleic acid is then detected. Detection may be accomplished by conventional separation techniques such as gel electrophoresis or by hybridization of at least a portion of a nucleotide probe comprising a nucleotide sequence complementary to the amplified HSV nucleic acid. The amplified HSV nucleic acid may also be detected by any suitable combination of detection techniques such as gel electrophoresis followed by hybridization with a nucleic acid probe. Preferably, however, amplified HSV-1 and/or HSV-2 (referred to interchangeably herein as ‘HSV-1/2’) nucleic acids are detected in a biological sample using real-time PCR techniques that can detect the increasing presence of an amplification product while amplification occurs.

“Nucleic acid” refers to a deoxyribonucleotide or ribonucleotide polymer in either single- or double-stranded form, and unless otherwise limited, would encompass known analogs of natural nucleotides that can function in a similar manner as naturally occurring nucleotides.

The term “oligonucleotide” refers to a molecule comprised of two or more deoxyribonucleotides or ribonucleotides, such as primers, probes, nucleic acid fragments to be detected, and nucleic acid controls. The exact size of an oligonucleotide depends on many factors and the ultimate function or use of the oligonucleotide.

The term “primer” refers to an oligonucleotide, whether natural or synthetic, capable of acting as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. A primer is preferably a single-stranded oligodeoxyribonucleotide. The appropriate length of a primer depends on the intended use of the primer but typically ranges from about 10 to about 30 nucleotides. Short primer molecules generally require cooler temperatures to form sufficiently stable hybrid complexes with the template. A primer need not reflect the exact sequence of the template but must be sufficiently complementary to specifically hybridize with a template. When primer pairs are referred to herein, the pair is meant to include one forward primer which is capable of hybridizing to the sense strand of a double-stranded target nucleic acid (the “sense primer”) and one reverse primer which is capable of hybridizing to the antisense strand of a double-stranded target nucleic acid (the “antisense primer”).

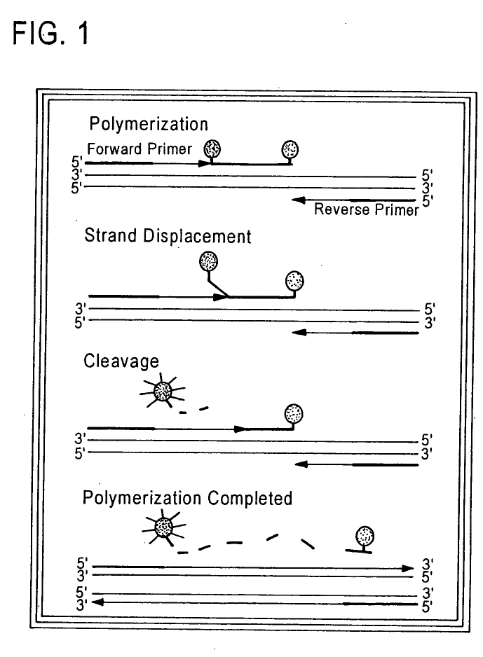

“Probe” refers to an oligonucleotide which binds through complementary base pairing to a sub-sequence of a target nucleic acid. A primer may be a probe. It will be understood by one of skill in the art that probes will typically substantially bind target sequences lacking complete complementarity with the probe sequence depending upon the stringency of the hybridization conditions. The probes are typically directly labeled (e.g., with isotopes or fluorescent moieties) or indirectly labeled such as with biotin to which a streptavidin complex may later bind. By assaying for the presence or absence of the probe, one can detect the presence or absence of the target, by Southern blot for example. Preferably, the target is an HSV-1 amplification product generated by PCR amplification of HSV-1 nucleic acids using primers SEQ ID NOs: 1 and 2, or oligonucleotides substantially identical thereto, respectively. Alternatively, the preferable target is an HSV-2 amplification product generated by PCR amplification of HSV-2 nucleic acids using primers SEQ ID NOs: 4 and 5, or oligonucleotides substantially identical thereto, respectively. More, preferably, the probes are fluorescently labeled and are capable of acting as a hydrolysis or Taqman® probes in a real-time PCR to amplify HSV nucleic acids. The real-time PCR method uses a dual labelled fluorogenic oligonucleotide probe (i.e., the hydrolysis or TaqMan® probe) that anneals specifically within the template amplicon spanning the forward (e.g. SEQ ID NO: 1 or SEQ ID NO: 4) and reverse primers (e.g. SEQ ID NO: 2 or SEQ ID NO: 5, respectively). Preferably, a fluorescent reporter molecule attached to 5′-position that fluoresces when released from probe by hydrolytic activity of DNA polymerase. Preferably, a quencher is attached to the 3′ end of the probe. A quencher is a fluorescent molecule that quenches (absorbs) the fluorescence emission from the Reporter only when Reporter is attached to the probe before hydrolysis. Preferably, laser stimulation within the capped wells containing the reaction mixture causes emission of a 3′ quencher dye (TAMRA) until the probe is cleaved by the 5′ to 3′ nuclease activity of the DNA polymerase during PCR extension, causing release of a 5′ reporter dye (e.g., 6FAM or ROX). Preferably, production of an amplicon causes emission of a fluorescent signal that is detected by a CCD (charge-coupled device) detection camera or other light capturing device, and the amount of signal produced at a threshold cycle within the purely exponential phase of the PCR reaction, reflects the starting copy number of the target sequence being amplified. See FIG. 1.

One aspect of the invention relates to oligonucleotide primers capable of acting as forward primers in a polymerase chain reaction (PCR) for amplifying an HSV-1 glycoprotein D gene. Preferably, the forward primer has the sequence: 5′-ATA CCG ACC ACA CCG ACG AA-3′ (SEQ ID NO: 1).

Another aspect of the invention relates to oligonucleotide primers capable of acting as reverse primers in a PCR reaction for amplifying an HSV-1 glycoprotein D gene. Preferably, the reverse primer has the sequence: 5′-ACG CAC CAC ACA AAA GAG ACC TT-3′ (SEQ ID NO: 2).

Another aspect of the invention relates to oligonucleotide primers capable of acting as forward primers in a polymerase chain reaction (PCR) for amplifying an HSV-2 glycoprotein G gene. Preferably, the forward primer has the sequence: 5′-CGC CAA ATA CGC CTT AGC A-3′ (SEQ ID NO: 4).

Another aspect of the invention relates to oligonucleotide primers capable of acting as reverse primers in a PCR reaction for amplifying an HSV-2 glycoprotein G gene. Preferably, the reverse primer has the sequence: 5′-GAA GGT TCT TCC CGC GAA AT-3′ (SEQ ID NO: 5).

Another aspect of the invention relates to oligonucleotides capable of acting as probe for the HSV-1 glycoprotein D gene. Preferably, the probe has the sequence: 5′-CTT CAG CGC GAA CGA CCA ACT AC-3′ (SEQ ID NO: 3).

Another aspect of the invention relates to oligonucleotides capable of acting as probe for the HSV-2 glycoprotein G gene. Preferably, the probe has the sequence: 5′-CCC CTC GCT TAA GAT GGC CG-3′ (SEQ ID NO: 6).

TABLE 1
Selected primer and probe sequences for PCR detection of HSV-1

| | | Position | | | | |
|---|---|---|---|---|---|---|
| Sequence | | in HSV-1 | | GC | Tₘ | Detection |
| Name | Nucleotide Sequence 5′ to 3′ | genome* | Length | (%) | (° C.) | System |
| HSV-1F | 5′-ATA CCG ACC ACA | 138,234 | 20- | 55.0% | 72.1 | |
| (forward) | CCG ACG AA -3′ | to | mer | | | |
| (SEQ ID | | 138,253 | | | | |
| NO: 1) | | | | | | |
| HSV-1R | 5′- ACG CAC CAC ACA | 138,390 | 23- | 47.8% | 72.7 | |
| (reverse) | AAA GAG ACC TT -3′ | to | mer | | | |
| (SEQ ID | | 138,413 | | | | |
| NO: 2) | | | | | | |
| HSV-1P | 5′-(Reporter)-CTT CAG CGC | 138,347 | 23- | 56.5% | 74.0 | Hydrolysis |
| probeA | GAA CGA CCA ACT AC - | to | mer | | | probe |
| (SEQ ID | (Quencher)-3′ | 138,370 | | | | |
| NO: 3) | | | | | | |

*GenBank Accession NC_001806

TABLE 2
Selected primer and probe sequences for PCR detection of HSV-2

| | | Position | | | | |
|---|---|---|---|---|---|---|
| Sequence | | in HSV-2 | | GC | Tₘ | Detection |
| Name | Nucleotide Sequence 5′ to 3′ | genome* | Length | (%) | (° C.) | System* |
| HSV-2F | 5′- CGC CAA ATA CGC CTT | 141,087 | 19- | 52.6% | 70.6 | |
| (forward) | AGC A -3′ | to | mer | | | |
| (SEQ ID | | 141,105 | | | | |
| NO: 4) | | | | | | |
| HSV-2R | 5′- GAA GGT TCT TCC CGC | 141,139 | 20- | 50.0% | 71.0 | |
| (reverse) | GAA AT -3′ | to | mer | | | |
| (SEQ ID | | 141,158 | | | | |
| NO: 5) | | | | | | |
| HSV-2P | 5′- (Reporter) CCC CTC GCT | 141,108 | 23- | 56.5% | 72.1 | Hydrolysis |
| probe | TAA GAT GGC CG | to | mer | | | probe |
| (SEQ ID | (Quencher)-3′ | 141,130 | | | | |
| NO: 6) | | | | | | |

* GenBank Accession NC_001798

The invention also includes oligonucleotides substantially identical to SEQ ID NOs: 1-6. For example, oligonucleotide sequences substantially identical to SEQ ID NOs: 1-6 may have several nucleotides added to, or removed from their 5′ ends or several nucleotides added to or removed from their 3′ ends. “Several nucleotides” in this context refers to about 3 nucleotides, or preferably about two nucleotides or more preferably about one nucleotide. The person of skill in the art will recognize that when adding nucleotides to the 5′ and/or 3′ ends SEQ ID NOs: 1-6, the identity of those nucleotides may be dictated by the sequence of the HSV-1 or HSV-2 DNA to be amplified. However, the skilled artisan may also wish to add overhangs to the 5′ end of the forward primer or 5′ end of the reverse primer (giving a 3′ sticky end on the amplicon). Such overhangs may include restriction enzyme sites useful in providing sticky ends to facilitate subcloning of the amplification product, for example.

Primers and probes of the invention exhibit an absence of hybridization to sequences contained in human RNA and DNA. This may be confirmed theoretically by BLAST analysis (NCBI), and empirically by testing selected primer sets against human total nucleic acid under both reverse transcription (RT) and/or PCR conditions. Additionally, the claims probes and primers lack cross reactivity against other non-HSV genomes that could be present in clinical samples. This may also be confirmed theoretically in a BLAST search, and empirically using for example, CMV, EBV, HHV6, HIV, HCV, HBV, enteroviral or parvoviral genomic material.

Primers and probes of the invention must be reactive with the dominant HSV-1 isolates circulating within the geographic region spanning the target patient population. Alternatively, primers and probes of the invention must be reactive with the dominant HSV-2 isolates circulating within the geographic region spanning the target patient population.

The skilled artisan will appreciate that the invention encompasses oligonucleotide sequences substantially identical to SEQ ID NOs: 1-6 where such sequences differ from SEQ ID NOs: 1-6, respectively, with respect to the identity of at least one nucleotide base yet retain the functionality of SEQ ID NOs: 1-6, respectively.

For example, one of skill in the art would envisage a genus of sequences substantially identical to SEQ ID NO: 1 wherein about one to about three nucleotides are added or removed from the 5′ end and/or about one to about three nucleotides are added or removed from the 3′ end, respectively, to include but not be limited to the following exemplary species:

TABLE 3

| | | |
|---|---|---|
| Seq. | Sequence Substantially Identical | |
| ID No. | to SEQ ID NO: 1 | Notes |

| | | |
|---|---|---|
| 7 | 5′- ATA CCG ACC ACA CCG ACG -3′ | 2 nt removed from ′3 end |
| 8 | 5′- A CCG ACC ACA CCG ACG AA -3′ | 2 nt removed from 5′ end |
| 9 | 5′- A CCG ACC ACA CCG ACG -3′ | 2 nt removed from 5′ end and 2 nt |
| | | removed from ′3 end |
| 10 | 5′- TA CCG ACC ACA CCG ACG A -3′ | 1 nt removed from 5′ end and 1 nt |
| | | removed from ′3 end |
| 11 | 5′- TA CCG ACC ACA CCG ACG AA -3′ | 1 nt removed from 5′ end |
| 12 | 5′- ATA CCG ACC ACA CCG ACG A -3′ | 1 nt removed from ′3 end |
| 13 | 5′- A CCG ACC ACA CCG ACG A -3′ | 2 nt removed from 5′ end and 1 nt |
| | | removed from ′3 end |
| 14 | 5′- TA CCG ACC ACA CCG ACG -3′ | 1 nt removed from 5′ end and 2 nt |
| | | removed from ′3 end |
| 15 | 5′- ATA CCG ACC ACA CCG ACG AAT C -3′ | 2 nt added to ′3 end |
| 16 | 5′- CC ATA CCG ACC ACA CCG ACG AA -3′ | 2 nt added to 5′ end |
| 17 | 5′- G\hat{ }AATTC ATA CCG ACC ACA CCG ACG AA -3′ | EcoRI site added to 5′ end |

Preferably, sequences substantially identical to SEQ ID NO: 1 have the same functionality as SEQ ID NO: 1 when used in a PCR reaction as a forward primer in conjunction with SEQ ID NO: 2 as a reverse primer. The following sequence is not considered to be encompassed in the present invention: 5′-CCGACCACACCGACGA-3′ (SEQ ID NO: 71) disclosed in JP 2002272499-A/5 which is hereby incorporated by reference in its entirety for all purposes.

Additionally, one of skill in the art would envisage a genus of sequences substantially identical to SEQ ID NO: 2 wherein about one to about three nucleotides are added or removed from the 5′ end and/or about one to about three nucleotides are added or removed from the 3′ end, respectively, to include but not be limited to the following exemplary species:

TABLE 4

| Seq. | Sequence Substantially Identical | |
|---|---|---|
| ID No. | to SEQ ID NO: 2 | Notes |
| 18 | 5′- ACG CAC CAC ACA AAA GAG AGC -3′ | 2 nt removed from ′3 end |
| 19 | 5′- G CAC CAC ACA AAA GAG AGG TT -3′ | 2 nt removed from 5′ end |
| 20 | 5′- G GAC CAC ACA AAA GAG ACC -3′ | 2 nt removed from 5′ end and 2 |
| | | nt removed from ′3 end |
| 21 | 5′- CG CAC CAC ACA AAA GAG ACC T -3′ | 1 nt removed from 5′ end and 1 |
| | | nt removed from ′3 end |
| 22 | 5′- CG CAC CAC ACA AAA GAG ACC TT -3′ | 1 nt removed from 5′ end |
| 23 | 5′- ACG CAC CAC ACA AAA GAG ACC T -3′ | 1 nt removed from ′3 end |
| 24 | 5′- G CAC CAC ACA AAA GAG ACC T -3′ | 2 nt removed from 5′ end and 1 |
| | | nt removed from ′3 end |
| 25 | 5′- CG CAC CAC ACA AAA GAG ACC -3′ | 1 nt removed from 5′ end and 2 |
| | | nt removed from ′3 end |
| 26 | 5′- ACG CAC CAC ACA AAA GAG ACC TTA A -3′ | 2 nt added to ′3 end |
| 27 | 5′- GA ACG CAC CAC ACA AAA GAG ACC TT -3′ | 2 nt added to 5′ end |
| 28 | 5′- G\hat{ }AATTC ACG CAC CAC ACA AAA GAG ACC TT -3′ | EcoRI site added to 5′ end |

Preferably, sequences substantially identical to SEQ ID NO: 2 have the same functionality as SEQ ID NO: 2 when used in a PCR reaction as a reverse primer in conjunction with SEQ ID NO: 1 as a forward primer.

One of skill in the art would envisage a genus of sequences substantially identical to SEQ ID NO: 3 wherein about one to about three nucleotides are added or removed from the 5′ end and/or about one to about three nucleotides are added or removed from the 3′ end, respectively, to include but not be limited to the following exemplary species:

TABLE 5

| Seq. | Sequence Substantially Identical | |
|---|---|---|
| ID No. | to SEQ ID NO: 3 | Notes |
| 29 | 5′- CTT CAG CGC GAA CGA CCA ACT -3′ | 2 nt removed from ′3 end |
| 30 | 5′- T CAG CGC GAA CGA CCA ACT AC -3′ | 2 nt removed from 5′ end |
| 31 | 5′- T CAG CGC GAA CGA CCA ACT -3′ | 2 nt removed from 5′ end and 2 |
| | | nt removed from ′3 end |
| 32 | 5′- TT CAG CGC GAA CGA CCA ACT A -3′ | 1 nt removed from 5′ end and 1 |
| | | nt removed from ′3 end |
| 33 | 5′- TT CAG CGC GAA CGA CCA ACT AC -3′ | 1 nt removed from 5′ end |
| 34 | 5′- CTT CAG CGC GAA CGA CCA ACT A -3′ | 1 nt removed from ′3 end |
| 35 | 5′- T CAG CGC GAA CGA CCA ACT A -3′ | 2 nt removed from 5′ end and 1 |
| | | nt removed from ′3 end |
| 36 | 5′- TT CAG CGC GAA CGA CCA ACT -3′ | 1 nt removed from 5′ end and 2 |
| | | nt removed from ′3 end |
| 37 | 5′- CTT CAG CGC GAA CGA CCA ACT ACC C -3′ | 2 nt added to ′3 end |
| 38 | 5′- AG CTT CAG CGC GAA CGA CCA ACT AC-3′ | 2nt added to 5′ end |

The following sequence is not considered to be encompassed in the present invention: 5′-AGCGCGAACGACCAACTACCCCGAT-3′ (SEQ ID NO: 72) disclosed in JP 995250699-A/21 which is hereby incorporated by reference in its entirety for all purposes.

One of skill in the art would envisage would envisage a genus of sequences substantially identical to SEQ ID NO: 4 wherein about one to about three nucleotides are added or removed from the 5′ end and/or about one to about three nucleotides are added or removed from the 3′ end, respectively, to include but not be limited to the following exemplary species:

TABLE 6

| | | |
|---|---|---|
| Seq. | Sequence Substantially Identical | |
| ID No. | to SEQ ID NO: 4 | Notes |

| | | |
|---|---|---|
| 39 | 5′- CGC CAA ATA CGC CTT AG -3′ | 2 nt removed from ′3 end |
| 40 | 5′- C CAA ATA CGC CTT AGC A -3′ | 2 nt removed from 5′ end |
| 41 | 5′- C CAA ATA CGC CTT AG -3′ | 2 nt removed from 5′ end and 2 nt |
| | | removed from ′3 end |
| 42 | 5′- GC CAA ATA CGC CTT AGC -3′ | 1 nt removed from 5′ end and 1 nt |
| | | removed from ′3 end |
| 43 | 5′- GC CAA ATA CGC CTT AGC A -3′ | 1 nt removed from 5′ end |
| 44 | 5′- CGC CAA ATA CGC CTT AGC -3′ | 1 nt removed from ′3 end |
| 45 | 5′- C CAA ATA CGC CTT AGC -3′ | 2 nt removed from 5′ end and 1 nt |
| | | removed from ′3 end |
| 46 | 5′- GC CAA ATA CGC CTT AG -3′ | 1 nt removed from 5′ end and 2 nt |
| | | removed from ′3 end |
| 47 | 5′- CGC CAA ATA CGC CTT AGC AGA-3′ | 2 nt added to ′3 end |
| 48 | 5′- TG CGC CAA ATA CGC CTT AGC A -3′ | 2 nt added to 5′ end |
| 49 | 5′-G\hat{ }AATTC CGC CAA ATA CGC CTT AGC A -3′ | EcoRI site added to 5′ end |

Preferably, sequences substantially identical to SEQ ID NO: 4 have the same functionality as SEQ ID NO: 4 when used in a PCR reaction as a forward primer in conjunction with SEQ ID NO: 5 as a reverse primer.

Additionally, one of skill in the art would envisage a genus of sequences substantially identical to SEQ ID NO: 5 wherein about one to about three nucleotides are added or removed from the 5′ end and/or about one to about three nucleotides are added or removed from the 3′ end, respectively, to include but not be limited to the following exemplary species:

TABLE 7

| Seq. | Sequence Substantially Identical | |
|---|---|---|
| ID No. | to SEQ ID NO: 5 | Notes |
| 50 | 5′- GAA GGT TCT TCC CGC GAA -3′ | 2 nt removed from ′3 end |
| 51 | 5′- A GGT TCT TCC CGC GAA AT -3′ | 2 nt removed from 5′ end |
| 52 | 5′- A GGT TCT TCC CGC GAA -3′ | 2 nt removed from 5′ end and 2 |
| | | nt removed from ′3 end |
| 53 | 5′- AA GGT TCT TCC CGC GAA A -3′ | 1 nt removed from 5′ end and 1 |
| | | nt removed from ′3 end |
| 54 | 5′- AA GGT TCT TCC CGC GAA AT -3′ | 1 nt removed from 5′ end |
| 55 | 5′- GAA GGT TCT TCC CGC GAA A -3′ | 1 nt removed from ′3 end |
| 56 | 5′- A GGT TCT TCC CGC GAA A -3′ | 2 nt removed from 5′ end and 1 |
| | | nt removed from ′3 end |
| 57 | 5′- AA GGT TCT TCC CGC GAA -3′ | 1 nt removed from 5′ end and 2 |
| | | nt removed from ′3 end |
| 58 | 5′- GAA GGT TCT TCC CGC GAA ATC G-3′ | 2 nt added to ′3 end |
| 59 | 5′- CG GAA GGT TCT TCG CGC GAA AT -3′ | 2 nt added to 5′ end |
| 60 | 5′- G\hat{ }AATTC GAA GGT TCT TCC CGC GAA AT -3′ | EcoRI site added to 5′ end |

Preferably, sequences substantially identical to SEQ ID NO: 5 have the same functionality as SEQ ID NO: 5 when used in a PCR reaction as a reverse primer in conjunction with SEQ ID NO: 4 as a forward primer.

One of skill in the art would envisage a genus of sequences substantially identical to SEQ ID NO: 6 wherein about one to about three nucleotides are added or removed from the 5′ end and/or about one to about three nucleotides are added or removed from the 3′ end, respectively, to include but not be limited to the following exemplary species:

TABLE 8

| Seq. | Sequence Substantially Identical | |
|---|---|---|
| ID No. | to SEQ ID NO: 6 | Notes |
| 61 | 5′- CCC CTC GCT TAA GAT GGC -3′ | 2 nt removed from ′3 end |
| 62 | 5′- C CTC GCT TAA GAT GGC CG -3′ | 2 nt removed from 5′ end |
| 63 | 5′- C CTC GCT TAA GAT GGC -3′ | 2 nt removed from 5′ end and 2 |
| | | nt removed from ′3 end |
| 64 | 5′- CC CTC GCT TAA GAT GGC C -3′ | 1 nt removed from 5′ end and 1 |
| | | nt removed from ′3 end |
| 65 | 5′- CC CTC GCT TAA GAT GGC CG -3′ | 1 nt removed from 5′ end |
| 66 | 5′- CCC CTC GCT TAA GAT GGC C -3′ | 1 nt removed from ′3 end |
| 67 | 5′- C CTC GCT TAA GAT GGC C -3′ | 2 nt removed from 5′ end and 1 |
| | | nt removed from ′3 end |
| 68 | 5′- CC CTC GCT TAA GAT GGC 3′ | 1 nt removed from 5′ end and 2 |
| | | nt removed from ′3 end |
| 69 | 5′- CCC CTC GCT TAA GAT GGC CGA T 3′ | 2 nt added to ′3 end |
| 70 | 5′-GA CCC CTC GCT TAA GAT GGC CG 3′ | 2 nt added to 5′ end |

Preferably, oligonucleotides sequences substantially identical to SEQ ID NOs: 1-6 will hybridize under stringent conditions (as defined herein) to all or a portion of the complements of SEQ ID NOs: 1-6 (i.e., target sequences), respectively. The terms “hybridize(s) specifically” or “specifically hybridize(s)” refer to complementary hybridization between an oligonucleotide (e.g., a primer or labeled probe) and a target sequence. The term specifically embraces minor mismatches that can be accommodated by reducing the stringency of the hybridization media to achieve the desired priming for the PCR polymerases or detection of hybridization signal.

Under stringent hybridization conditions, only highly complementary, i.e., substantially identical nucleic acid sequences, hybridize. Preferably, such conditions prevent hybridization of nucleic acids having 3 or more mismatches out of 20 contiguous nucleotides, more preferably 2 or more mismatches out of 20 contiguous nucleotides, most preferably one or more mismatch out of 20 contiguous nucleotides. The hybridizing portion of the hybridizing nucleic acid is at least about 90%, preferably at least about 95%, or most preferably about at least about 98%, identical to the sequence of a target sequence, or its complement.

Hybridization of a nucleic acid to a nucleic acid sample under stringent conditions is defined below. Nucleic acid duplex or hybrid stability is expressed as a melting temperature (Tₘ), which is the temperature at which the probe dissociates from the target DNA. This melting temperature is used to define the required stringency conditions. If sequences are to be identified that are substantially identical to the probe, rather than identical, then it is useful to first establish the lowest temperature at which only homologous hybridization occurs with a particular concentration of salt (e.g. SSC or SSPE). Then assuming that 1% mismatching results in a 1° C. decrease in Tₘ, the temperature of the final wash in the hybridization reaction is reduced accordingly (for example, if sequences having >95% identity with the probe are sought, the final wash temperature is decrease by 50° C.). In practice, the change in Tₘ can be between 0.5° C. and 1.5° C. per 1% mismatch.

Stringent conditions involve hybridizing at 68° C. in 5×SSC/5× Denhart's solution/1.0% SDS, and washing in 0.2×SSC/0.1% SDS at room temperature. Moderately stringent conditions include washing in 3×SSC at 42° C. The parameters of salt concentration and temperature may be varied to achieve optimal level of identity between the primer and the target nucleic acid. Additional guidance regarding such conditions is readily available in the art, for example, Sambrook, Fischer and Maniatis, Molecular Cloning, a laboratory manual, (2nd ed.), Cold Spring Harbor Laboratory Press, New York, (1989) and F. M. Ausubel et al eds., Current Protocols in Molecular Biology, John Wiley and Sons (1994).

Another aspect of the invention relates to a method of detecting HSV-1 DNA by using SEQ ID NOs: 1 and 2; or oligonucleotides substantially identical thereto, in a polymerase chain reaction performed on a biological sample. Another aspect of the invention relates to a method of detecting HSV-2 DNA by using SEQ ID NOs: 4 and 5; or oligonucleotides substantially identical thereto, in a polymerase chain reaction performed on a biological sample.

Another aspect of the invention relates to a kit for detecting HSV-1 DNA having SEQ ID NOs: 1 and 2 or oligonucleotides substantially identical thereto. One embodiment of this aspect of the invention utilizes real-time PCR and includes at least one probe e.g. SEQ ID NO: 3, or oligonucleotides substantially identical thereto. Another aspect of the invention relates to a kit for detecting HSV-2 having SEQ ID NOs: 4 and 5 or oligonucleotides substantially identical thereto. One embodiment of this aspect of the invention utilizes real-time PCR and includes at least one probe e.g. SEQ ID NO: 6, or oligonucleotides substantially identical thereto.

The present methods and oligonucleotides can be applied to any type of biological sample that is suspected of containing HSV-1/2 DNA. The term “biological sample” refers to a sample comprising any biological material (e.g., biological fluids or tissues) containing nucleic acids. Biological samples can include tissue samples, whole blood or serum, sputum, stool, urine, semen, pericardial fluid, nasopharyngeal/throat swabs, cerebrospinal fluid (CSF), amniotic fluid and the like. Preferably, the biological sample is CSF or blood serum. Tissues may, for example, be surgically resected from a patient in the form of a biopsy or autopsy tissue sample. Preferably, at least about 50 mg of tissue is resected. Preferably, all biological samples are transported at room temperature for overnight shipping and immediate processing. All bodily fluid biological samples (other than whole blood) may be stored frozen if processed at a later time.

Due to the different clinical presentations of HSV infection, DNA extraction protocols used for molecular testing have been optimized for multiple specimen types including blood (0.2 to 0.5 cc), cerebral spinal fluid (CSF, 0.2 cc), urine, vesicular lesion scraping/fluid, and swab specimens (eye, ear, skin, rectal, nasopharyngeal).

In one embodiment where blood is the biological sample, peripheral blood collected in EDTA blood tube (1-3 ml). For neonates, about 0.5-1.0 ml peripheral or heel-stick blood acceptable. In special cases where blood is limiting (premature births), about 0.2 ml is the minimal acceptable volume. In another embodiment where cerebral spinal fluid is the biological sample, a 1.0 ml volume of CSF collected in a sterile collection tube. For newborns, about 0.2-0.5 ml is acceptable. In another embodiment where pericardial fluid is the biological sample about 1.0 ml volume, or more, of PF is preferred. In another embodiment where amniotic fluid is the biological sample, a 2-3 ml volume of whole AF is acceptable. AF supernatant (cells removed by centrifugation) is also acceptable for enteroviral RT-PCR testing (see U.S. Ser. No. 10/829,474, which is herein incorporated by reference). In another embodiment where nasopharyngeal and throat swabs are the biological sample, the swabs are in the form of Dacron collection swab in about 3.0 ml of M4 transport media. In another embodiment where tissue samples are the biological sample, about 25 mg of biopsy material is quick-frozen and placed in sterile container.

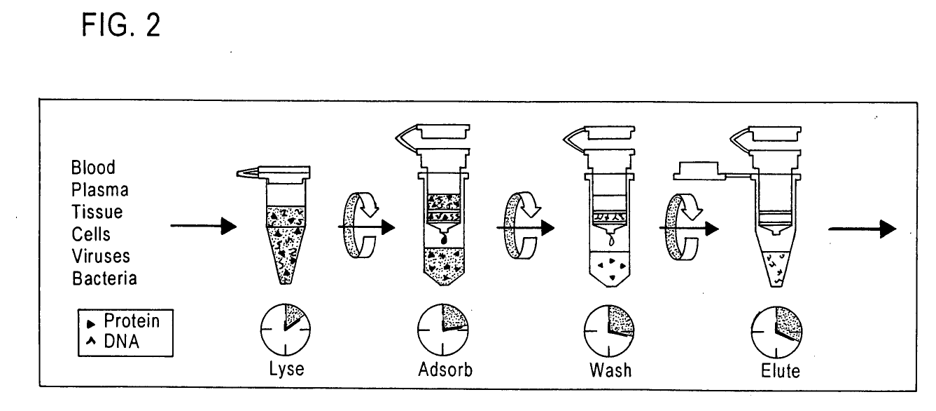

Since the quality of the molecular data is related to the quality of prepared template DNA, all specimens are preferably processed using detergent lysis with Proteinase K digestion followed by purification on silica-based nucleic acid binding columns (FIG. 2). Pure DNA is recovered within 30-45 minutes and ready for molecular amplification.

To amplify a target nucleic acid sequence in a biological sample by PCR, the sequence must be accessible to the components of the amplification system. In general, this accessibility is ensured by isolating the nucleic acids from the sample.

Preferably, the methods of the invention are performed with total DNA isolated from the biological sample, as the starting material. A variety of techniques for extracting nucleic acids, from biological samples are known in the art. Alternatively, if the sample is fairly readily disruptable, the nucleic acid may not need to be purified prior to amplification by the PCR technique, i.e., if the sample is comprised of cells, particularly peripheral blood lymphocytes or monocytes, lysis and dispersion of the intracellular components may be accomplished merely by suspending the cells in hypotonic buffer.

If it is not possible to extract DNA from the tissue sample soon after its resection, the sample may be fixed or frozen. DNA extracted and isolated from frozen or fresh samples of resected tissue is extracted by any method known in the art, for example, Sambrook, Fischer and Maniatis, Molecular Cloning, a laboratory manual, (2nd ed.), Cold Spring Harbor Laboratory Press, New York, (1989). Preferably, care is taken to avoid degradation of DNA during the extraction process.

Alternatively, tissue obtained from the patient may be fixed, preferably by formalin (formaldehyde) or gluteraldehyde treatment, for example. Biological samples fixed by alcohol immersion are also contemplated in the present invention. Fixed biological samples are often dehydrated and embedded in paraffin or other solid supports known to those of skill in the art. Such solid supports are envisioned to be removable with organic solvents, allowing for subsequent rehydration of preserved tissue. Fixed and paraffin-embedded (FPE) tissue sample as described herein refers to storable or archival tissue samples.

DNA may be extracted from a frozen or FPE sample by any of the methods as described in U.S. Pat. No. 6,428,963, which is hereby incorporated by reference in its entirety. In one embodiment of the invention, DNA is isolated from an archival pathological sample or biopsy which is first deparaffinized. An exemplary deparaffinization method involves washing the paraffinized sample with an organic solvent, such as xylene. Deparaffinized samples can be rehydrated with an aqueous solution of a lower alcohol. Suitable lower alcohols, for example include, methanol, ethanol, propanols, and butanols. Deparaffinized samples may be rehydrated with successive washes with lower alcoholic solutions of decreasing concentration. Alternatively, the sample is simultaneously deparaffinized and rehydrated.

Once the sample is reyhdrated, DNA is extracted and isolated from the rehydrated tissue. Deparaffinized samples can be homogenized using mechanical, sonic or other means of homogenization, e.g. by laser microdisection. In one embodiment, rehydrated samples are homogenized in a solution comprising a chaotropic agent, such as guanidinium thiocyanate (also sold as guanidinium isothiocyanate).

Chaotropic agents include but not limited to: guanidinium compounds, urea, formamide, potassium iodiode, potassium thiocyantate and similar compounds. The preferred chaotropic agent for the methods of the invention is a guanidinium compound, such as guanidinium isothiocyanate (also sold as guanidinium thiocyanate) and guanidinium hydrochloride. Many anionic counterions are useful, and one of skill in the art can prepare many guanidinium salts with such appropriate anions. The effective concentration of guanidinium solution used in the invention generally has a concentration in the range of about 1 to about 5M with a preferred value of about 4M. If DNA is already in solution, the guanidinium solution may be of higher concentration such that the final concentration achieved in the sample is in the range of about 1 to about 5M. The guanidinium solution also is preferably buffered to a pH of about 3 to about 6, more preferably about 4, with a suitable biochemical buffer such as Tris-Cl. The chaotropic solution may also contain reducing agents, such as dithiothreitol (DTT), (β-mercaptoethanol; BME); and combinations thereof. The chaotropic solution may also contain DNAse and/or RNAse inhibitors.

DNA is then recovered from the solution by, for example, phenol chloroform extraction, ion exchange chromatography or size-exclusion chromatography. DNA may then be further purified using the techniques of extraction, electrophoresis, chromatography, precipitation or other suitable techniques.

The amplification of HSV-1/2 DNA is preferably carried out using polymerase chain reaction (PCR) methods common in the art. The first step of each cycle of the PCR involves the separation of the nucleic acid duplex formed by the primer extension. Once the strands are separated, the next step in PCR involves hybridizing the separated strands with primers that flank the target sequence e.g. SEQ ID NOs: 1 or 4 and 2 or 5, respectively. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid. Strand separation is achieved by heating the reaction to a sufficiently high temperature for a sufficient time to cause the denaturation of the duplex but not to cause an irreversible denaturation of the polymerase (see U.S. Pat. No. 4,965,188). Template-dependent extension of primers in PCR is catalyzed by a polymerizing agent in the presence of adequate amounts of four deoxyribonucleoside triphosphates (typically dATP, dGTP, dCTP, and dTTP) in a reaction medium comprised of the appropriate salts, metal cations, and pH buffering system. Suitable polymerizing agents are enzymes known to catalyze template-dependent DNA synthesis. For example, Thermus thermophilus (Tth) DNA polymerase, a thermostable DNA polymerase with reverse transcriptase activity is marketed by Roche Molecular Systems (Alameda, Calif.) PCR is most usually carried out as an automated process with a thermostable enzyme. In this process, the temperature of the reaction mixture is cycled through a denaturing region, a primer annealing region, and an extension reaction region automatically. Equipment specifically adapted for this purpose is commercially available from Roche Molecular Systems.

Most preferably, amplification of HSV-1/2 DNA is carried out using a fluorescence based real-time detection method (e.g. SmartCycler®, Cepheid, or the ABI PRISM 7700 or 7900 Sequence Detection System [TaqMan®], Applied Biosystems, Foster City, Calif.) or similar system as described by Heid et al., (Genome Res 1996;6:986-994) and Gibson et al.(Genome Res 1996;6:995-1001). The output of the ABI 7700 (TaqMan® Instrument) is expressed in Ct's or “cycle thresholds”. A higher number of target molecules in a sample generates a signal with fewer PCR cycles (lower Ct) than a sample with a lower number of target molecules (higher Ct). By extension, given a set number of cycles, the level of fluorescence generated in the reaction will be indicative of the amount of amplification product which in turn, is a function of the amount template nucleic acid in the original biological sample. Therefore, real-time PCR also allows for the quantification of template DNA in the original biological sample. Preferably, the hydrolysis or TaqMan® probes for the oligonucleotide primer pair SEQ ID NO: 1 and 2, is SEQ ID NOs: 3; and the hydrolysis or TaqMan® probes for the oligonucleotide primer pair SEQ ID NO: 4 and 5 is SEQ ID NOs: 6.

In one aspect of the current invention, a PCR is set up to simultaneously amplify a portion of a plurality of nucleic acid templates including the HSV-1 glycoprotein D gene and HSV-2 glycoprotein G gene. As such, the reaction includes SEQ ID NOs 1 and 2 or oligonucleotides substantially identical thereto; as well as SEQ ID NOs: 4 and 5, or oligonucleotides substantially identical thereto. A PCR device such as the Taqman® device, can discern between the amplification products resulting from the parallel reactions by way of the differing reporter dyes associated with their respective probes in the reaction. Preferably, the HSV-1 probe (HSV-1P; SEQ ID NO: 3) and the HSV-2 probe (HSV-2P; SEQ ID NO: 6) are associated with 6FAM and ROX, respectively. This is referred to the art as multiplex PCR. However, the skilled artisan will appreciate that there are a number of other suitable dyes available.

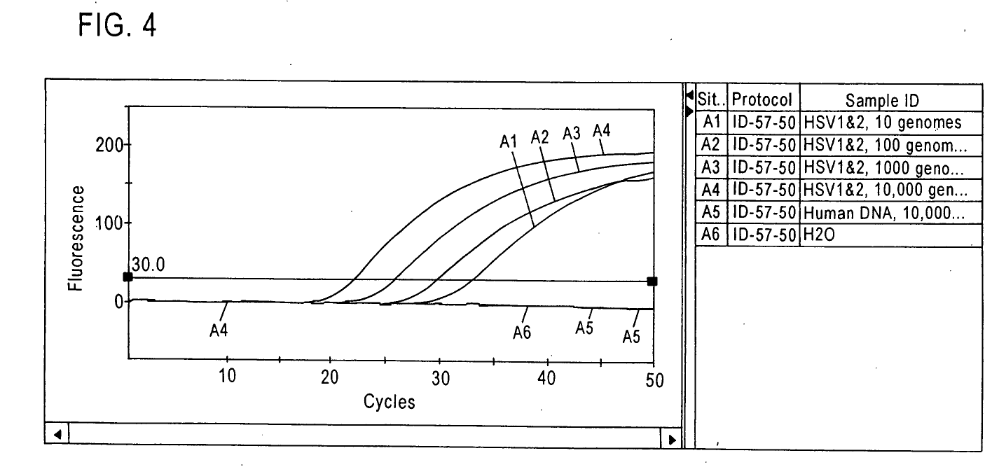

In this embodiment, the HSV-1 glycoprotein D (gD) gene is targeted for amplification and for HSV-2 the target is the glycoprotein G (gG) gene. Unlike “nested” PCR that uses 2 rounds of amplification to achieve the required sensitivity with 2 different primer sets for specificity, real-time PCR utilizes a single round of PCR using I primer set in a closed-tube format, reducing tube transfers, sample switching, and end-product contamination. Included in the amplification reaction is an amplicon-specific, fluorogenic hybridization, e.g., Taqman®, probe that signals the presence of amplicon. When amplicon, and therefore target template, is present the probe binds the nascent amplicon and is converted from a “dark” state to a fluorescent state via probe hydrolysis in direct proportion to the amount of amplicon present. For signal detection, real-time thermocyclers employ 2 optical components, the first component introduces light into the reaction tube at the excitation wavelengths for the reported fluorophore(s), and a second component detects emission fluorescence, typically at 4 different spectra for multiplex assays. The property of fluorescence accumulation in proportion to amplicon concentration makes possible quantitative testing when clinical outcomes are compared to a standard curve run in parallel. Quantitative PCR testing is therefore possible for all the pathogen assays formatted for real-time, as long as appropriate known standards are available for a given agent and that some form of standardization of data is possible between labs (FIGS. 4 & 5).

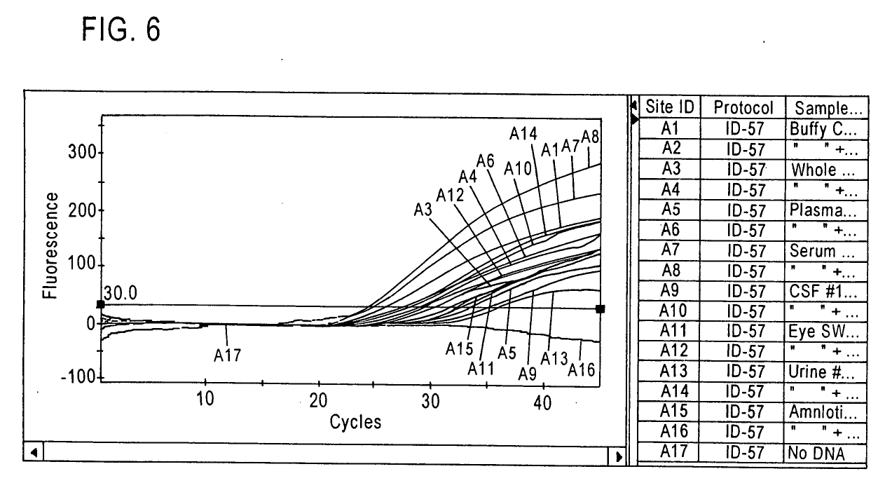

In one embodiment, the assays include of a primer set and probe specific for a DNA sequence specific to the host from which the biological sample is taken, to serve as an internal positive control. Preferably, this patient-derived target sequence is scored as positive, indicating that nucleic acid extraction, enzymatic amplification, and detection chemistry proceeded normally, before a determination can be made on the presence or absence of pathogenic target (FIG. 6). Most preferably a positive control experiment is carried out in parallel with the assay herein, ensuring that a false-negative due to suboptimal assay performance is not mistaken for a “true” negative result. Even more preferably, with the use of an internal positive control, the HSV assay is a 3-part multiplex containing primer sets specific for HSV-1, HSV-2, and patient target sequence, with 3 amplicon-specific probes all contained in a single master mix. With multi-color detection capability, each HSV serotype and the human target have individual detection wavelengths, which are assessed and displayed independently for each channel.

In summary, 2 types of amplification controls can be employed in this assay design: 1) an internal patient-derived control involving the detection of a human sequence from the patient, and 2) a second, parallel reaction identical to first except for the inclusion of a “spike” of uninfected human DNA. The primers and probe specific for this human sequence will generate a signal in any reaction containing human DNA as long as the amplification reaction is not inhibited. Typically, the human sequence is detected in both reactions, indicating no inhibition. The detection of signal in the first “unspiked” sample indicates that human DNA was successfully recovered from the specimen. The second “spiked” sample is informative for specimens containing very few human cells, such as CSF and serum, in which case a patient-derived signal would not be expected. In this situation, the detection of the human sequence from the parallel “spiked” sample is required in order to eliminate the possibility of enzyme inhibition, since any biological inhibitors would be found in both samples since they contain the same amount of patient extract. In the rare case when neither reaction displays the human internal control signal indicating failure of amplification, the assay is repeated using a second specimen.

In one embodiment, the use of a PCR program consisting of about 45 to about 50 cycles lasting about 45 seconds each, a real-time analysis is complete within about 50 minutes. With this program, all assays have about a 10-genome detection threshold for the viral genetic targets included in the multiplex. Preferably, by combining specimen preparation and molecular amplification/detection, up to about 96 samples can be completed in under about 2 hours from receipt of specimens in the lab with all fluorescent data displayed in real-time and electronically analyzed and stored. In cases of strong viral positives, detection can be confirmed before the analysis is complete and reported, if clinically significant. The 10-genome detection threshold for the assay translates to the detection of about 250 viral genomes per 1 ml of CSF or plasma. Higher sensitivity can be achieved by including more of the patient extract in the amplification reaction, but this increases the chances of reaction inhibition, and the above-mentioned threshold is considered clinically relevant.

One of skill will recognize, however, that the oligonucleotides of the invention are useful for detecting HSV-1/2 DNA by any known method, such as ligase chain reaction (LCR) or self-sustained sequence replication, each of which provides sufficient amplification. Additionally, the present invention envisages the quantification of HSV-1/2 DNA via use of a PCR-free systems employing, for example fluorescent labeled probes similar to those of the Invader® Assay (Third Wave Technologies, Inc.).

Another aspect of the invention relates to a method of identifying compounds capable of inhibiting HSV-1/2. The method generally entails infecting a tissue culture with a herpes simplex virus and then contacting a portion of the infected tissue culture with a compound suspected of being capable of inhibiting HSV growth. Next nucleic acids are isolated from the portion of the infected tissue culture contacted by the candidate compound. As a control, nucleic acids are also isolated from a portion of the remainder of the infected tissue culture not contacted by the candidate compound. Next, PCR is performed on both nucleic acid samples in parallel. Preferably, SEQ ID NO: 1 or an oligonucleotide substantially identical thereto is used as the forward primer and SEQ ID NO: 2 or an oligonucleotide substantially identical thereto is used as the reverse primer, when assaying for the presence and/or amount of HSV-1 DNA. Preferably, SEQ ID NO: 4 or an oligonucleotide substantially identical thereto is used as the forward primer and SEQ ID NO: 5 or an oligonucleotide substantially identical thereto is used as the reverse primer, when assaying for the presence and/or amount of HSV-2 DNA. A decrease in an amplification product in the nucleic acid sample derived from the treated tissue culture relative to an amount of amplification product in the nucleic acid sample derived from to the control indicates that a candidate compound is capable of inhibiting HSV growth. Preferably, tissue culture comprises cells derived from the group consisting of African Green Money Vero cells, Human HEp-2 cells (refs. Smith K O. Relationship between the envelope and the infectivity of herpes simplex virus. Proc. Soc. Exp. Biol. Med. 115: 814-816, 1964. and Willard M. Rapid directional translocations in virus replication. J. Virol. 76: 5220-5232, 2002.), and/or new born mice. Additionally, the term “tissue culture” as used herein is not limited to in vitro uses. The term also encompasses live animals that act as incubators for HSV growth such as suckling mice, rats or other mammals.

### EXAMPLE 1

Real-time PCR Procedure for HSV-1/2 Detection

Preparation of template DNA: For HSV-1/2 PCR, 200-ul of cerebral spinal fluid (CSF), whole blood, serum, plasma, pericardial fluid, or of media recovered from swabs, etc., is processed using any appropriate nucleic extraction protocol, such as the Qiagen DNA mini kit, or the Gentra Puregene DNA purification kit, according to the manufacturers instructions. Final nucleic acid is recovered using a 60-100-ul volume of sterile water, or appropriate PCR compatible buffer. A 5.0-ul volume of nucleic acid extract is used for a 25-ul PCR.

Amplification: Fluorochromes for HSV-1P (SEQ ID NO: 3) and HSV-2P (SEQ ID NO: 6) are of different emission wavelengths to achieve single-tube detection and serotype differentiation. FAM was used for HSV-1P and ROX for HSV-2P. PCR performed with MgCl₂ at 4 mM; dNTPs at 0.2 mM and 1 unit of Taq per reaction, or 0.04 U/μl, final concentration. Exemplary reaction conditions are outlined in the table below:

A 10× mix of the HSV 1 (SEQ ID NOs: 1, 2 and 3) & HSV-2 (SEQ ID NOs: 4, 5 and 6) primers and probes to give the final working concentrations for (5) 25 μL reactions. A single reaction with 5.0 μL of sample template was be prepared as follows:

TABLE 9

| Reagent | stock | conc | Vol, μL | final | cone | Lot # |
|---|---|---|---|---|---|---|
| dH2O | | | 12.55 | | | |
| 10× PCR | 10 | X | 2.5 | 1 | X | |
| buffer | | | | | | |
| MgCl2 | 50 | mM | 2.0 | 4.0 | mM | |
| dNTPs | 20 | mM | 0.25 | 0.2 | mM | |
| HSV1&2 | 10 | X | 2.5 | 1 | X | |
| mix | | | | | | |
| Taq Pol | 5 | U/μl | 0.2 | 0.04 | U/μl | |

Below are the PCR conditions used:

TABLE 10

| Cycles | Stage | Temperature | Time | |
|---|---|---|---|---|
| 1 | Hot start | 94° C. | 120 | sec |
| | Denature | 95° C. | 15 | seconds |
| 45 | Anneal | 57° C. | 15 | seconds |
| | Extend | 72° C. | 15 | seconds |
| 1 | Melt | 60° C. to 95° C. | 0.2° | C./sec |

The optics were turned on during the anneal step.

In this disclosure there are described only the preferred embodiments of the invention and but a few examples of its versatility. It is to be understood that the invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances and procedures described herein. Such equivalents are considered to be within the scope of this invention.

What is claimed is:

1. An isolated oligonucleotide of the sequence SEQ ID NO: 1.

2. An isolated oligonucleotide that hybridizes the complement of SEQ ID NO: 1 under stringent conditions and is capable of amplifying an HSV-1 glycoprotein D gene when used in conjunction with SEQ ID NO: 2 in a polymerase chain reaction.

3. An isolated oligonucleotide of the sequence of SEQ ID NO: 1, wherein from about one to about three nucleotides are added or removed from the 5′ end and/or from about one to about three nucleotides are added or removed from the 3′ end, respectively, and wherein the oligonucleotide is capable of amplifying an HSV-1 glycoprotein D gene when used in conjunction with SEQ ID NO: 2 in an polymerase chain reaction.

4. An isolated oligonucleotide of the sequence SEQ ID NO: 2.

5. An isolated oligonucleotide that hybridizes the complement of SEQ ID NO: 2 under stringent conditions and is capable of amplifying an HSV-1 glycoprotein D gene when used in conjunction with SEQ ID NO: 1 in a polymerase chain reaction.

6. An isolated oligonucleotide of the sequence of SEQ ID NO: 2, wherein from about one to about three nucleotides are added or removed from the 5′ end and/or from about one to about three nucleotides are added or removed from the 3′ end, respectively, and wherein the oligonucleotide is capable of amplifying an HSV-1 glycoprotein D gene when used in conjunction with SEQ ID NO: 1 in an polymerase chain reaction.

7. An isolated oligonucleotide having the sequence of SEQ ID NO: 3 or a sequence wherein about one to about three nucleotides are added or removed from the 5′ end and/or about one to about three nucleotides are added or removed from the 3′ end of SEQ ID NO: 3.

8. A kit for detecting HSV-1 comprising a first isolated oligonucleotide of SEQ ID NO: 1 and a second oligonucleotide of any one of claims 4 to 6.

9. A kit for detecting HSV-1 comprising a first isolated oligonucleotide of SEQ ID NO: 2 and a second oligonucleotide of any one of claims 1 to 3.

10. A kit for detecting HSV-1 comprising a first isolated oligonucleotide of SEQ ID NO: 1 and a second oligonucleotide of SEQ ID NO: 2.

11. A kit for detecting HSV-1 DNA comprising a first oligonucleotide selected from the group consisting of:
(A) an isolated oligonucleotide of the sequence SEQ ID NO: 1;
(B) an isolated oligonucleotide that hybridizes the complement of SEQ ID NO: 1 under stringent conditions and is capable of amplifying an HSV-1 glycoprotein D gene when used in conjunction with SEQ ID NO: 2 in an polymerase chain reaction; and
(C) an isolated oligonucleotide of the sequence of SEQ ID NO: 1, wherein from about one to about three nucleotides are added or removed from the 5′ end and/or from about one to about three nucleotides are added or removed from the 3′ end, respectively, and wherein the oligonucleotide is capable of amplifying an HSV-1 glycoprotein D gene when used in conjunction with SEQ ID NO: 2 in an polymerase chain reaction;
and a second oligonucleotide selected from the group consisting of
(A) an isolated oligonucleotide of the sequence SEQ ID NO: 2;
(B) an isolated oligonucleotide that hybridizes the complement of SEQ ID NO: 2 under stringent conditions and is capable of amplifying an HSV-1 glycoprotein D gene when used in conjunction with SEQ ID NO: 1 in an polymerase chain reaction; and
(C) an isolated oligonucleotide of the sequence of SEQ ID NO: 2, wherein from about one to about three nucleotides are added or removed from the 5′ end and/or from about one to about three nucleotides are added or removed from the 3′ end, respectively, and wherein the oligonucleotide is capable of amplifying an HSV-1 glycoprotein D gene when used in conjunction with SEQ ID NO: 1 in an polymerase chain reaction.

12. The kit of claims 10 or 11 further comprising SEQ ID No: 3 or a sequence wherein about one to about three nucleotides are added or removed from the 5′ end and/or about one to about three nucleotides are added or removed from the 3′ end of the sequence set forth in SEQ ID NO: 3.

13. The kit of claims 10 or 11 further comprising a PCR reaction buffer and DNA polymerase enzyme.

14. A method of detecting the presence of HSV-1 in a biological sample comprising:
(A) obtaining a biological sample from an organism;
(B) isolating nucleic acids from said sample;
(C) performing a polymerase chain reaction on said isolated nucleic acids using a first isolated oligonucleotide selected from the group consisting of:
(i) an isolated oligonucleotide of the sequence SEQ ID NO: 1;
(ii) an isolated oligonucleotide that hybridizes the complement of SEQ ID NO: 1 under stringent conditions and is capable of amplifying an HSV-1 glycoprotein D gene when used in conjunction with SEQ ID NO: 2 in an polymerase chain reaction; and
(iii) an isolated oligonucleotide of the sequence of SEQ ID NO: 1, wherein from about one to about three nucleotides are added or removed from the 5′ end and/or from about one to about three nucleotides are added or removed from the 3′ end, respectively, and wherein the oligonucleotide is capable of amplifying an HSV-1 glycoprotein D gene when used in conjunction with SEQ ID NO: 2 in an polymerase chain reaction;
and a second oligonucleotide selected from the group consisting of
(i) an isolated oligonucleotide of the sequence SEQ ID NO: 2;
(ii) an isolated oligonucleotide that hybridizes the complement of SEQ ID NO: 2 under stringent conditions and is capable of amplifying an HSV-1 glycoprotein D gene when used in conjunction with SEQ ID NO: 1 in an polymerase chain reaction; and
(iii) an isolated oligonucleotide of the sequence of SEQ ID NO: 2, wherein from about one to about three nucleotides are added or removed from the 5′ end and/or from about one to about three nucleotides are added or removed from the 3′ end, respectively, and wherein the oligonucleotide is capable of amplifying an HSV-1 glycoprotein D gene when used in conjunction with SEQ ID NO: 1 in an polymerase chain reaction,
(D) correlating a presence of an amplification product from said polymerase chain reaction with the presence of HSV-1 in said sample.

15. The method of claim 14 wherein, the biological sample is selected from the group consisting of a tissue sample, whole blood or serum, sputum, stool, urine, semen, pericardial fluid, nasopharyngeal/throat swabs, cerebrospinal fluid (CSF), and amniotic fluid.

16. The method 14 wherein the organism is a patient suspected of having being infected by an HSV-1.

17. The method of claim 14 where the polymerase chain reaction is a real-time polymerase chain reaction.

18. The method of identifying compounds capable of inhibiting HSV-1 growth comprising:
(A) infecting a tissue culture with an HSV-1 to obtain an infected tissue culture;
(B) contacting a portion of said infected tissue culture with a compound suspected of being capable of inhibiting HSV-1 growth;
(C) isolating nucleic acids from the portion of said infected tissue culture contacted by said compound to obtain a first nucleic acid sample and from a portion of the remainder of the infected tissue culture not contacted by said compound to obtain a second nucleic acid sample;
(D) performing polymerase chain reaction on said first and said second nucleic acid samples, using a first isolated oligonucleotide selected from the group consisting of:
(i) an isolated oligonucleotide of the sequence SEQ ID NO: 1;
(ii) an isolated oligonucleotide that hybridizes the complement of SEQ ID NO: 1 under stringent conditions and is capable of amplifying an HSV-1 glycoprotein D gene when used in conjunction with SEQ ID NO: 2 in an polymerase chain reaction; and
(iii) an isolated oligonucleotide of the sequence of SEQ ID NO: 1, wherein from about one to about three nucleotides are added or removed from the 5′ end and/or from about one to about three nucleotides are added or removed from the 3′ end, respectively, and wherein the oligonucleotide is capable of amplifying an HSV-1 glycoprotein D gene when used in conjunction with SEQ ID NO: 2 in an polymerase chain reaction;
and a second oligonucleotide selected from the group consisting of
(i) an isolated oligonucleotide of the sequence SEQ ID NO: 2;
(ii) an isolated oligonucleotide that hybridizes the complement of SEQ ID NO: 2 under stringent conditions and is capable of amplifying an HSV-1 glycoprotein D gene when used in conjunction with SEQ ID NO: 1 in an polymerase chain reaction; and
(iii) an isolated oligonucleotide of the sequence of SEQ ID NO: 2, wherein from about one to about three nucleotides are added or removed from the 5′ end and/or from about one to about three nucleotides are added or removed from the 3′ end, respectively, and wherein the oligonucleotide is capable of amplifying an HSV-1 glycoprotein D gene when used in conjunction with SEQ ID NO: 1 in an polymerase chain reaction,
(D) whereby a decrease in an amplification product in the first nucleic acid sample relative to the second nucleic acid sample indicates that the compound is capable of inhibiting HSV-1 growth.

19. The method of claim 18 wherein said tissue culture comprises cells derived from the group consisting of African Green Money (Vero cells), Human HEp-2 cells, and new born mice.

20. An isolated oligonucleotide of the sequence SEQ ID NO: 4.

21. An isolated oligonucleotide that hybridizes the complement of SEQ ID NO: 4 under stringent conditions and is capable of amplifying an HSV-2 glycoprotein G gene when used in conjunction with SEQ ID NO: 5 in a polymerase chain reaction.

22. An isolated oligonucleotide of the sequence of SEQ ID NO: 4, wherein from about one to about three nucleotides are added or removed from the 5′ end and/or from about one to about three nucleotides are added or removed from the 3′ end, respectively, and wherein the oligonucleotide is capable of amplifying an HSV-2 glycoprotein G gene when used in conjunction with SEQ ID NO: 5 in an polymerase chain reaction.

23. An isolated oligonucleotide of the sequence SEQ ID NO: 5.

24. An isolated oligonucleotide that hybridizes the complement of SEQ ID NO: 5 under stringent conditions and is capable of amplifying an HSV-2 glycoprotein G gene when used in conjunction with SEQ ID NO: 4 in an polymerase chain reaction.

25. An isolated oligonucleotide of the sequence of SEQ ID NO: 5, wherein from about one to about three nucleotides are added or removed from the 5′ end and/or from about one to about three nucleotides are added or removed from the 3′ end, respectively, and wherein the oligonucleotide is capable of amplifying an HSV-2 glycoprotein G gene when used in conjunction with SEQ ID NO: 4 in an polymerase chain reaction.

26. An isolated oligonucleotide having the sequence of SEQ ID NO: 6 or a sequence wherein about one to about three nucleotides are added or removed from the 5′ end and/or about one to about three nucleotides are added or removed from the 3′ end of SEQ ID NO: 6.

27. A kit for detecting HSV-2 comprising a first isolated oligonucleotide of SEQ ID NO: 4 and a second oligonucleotide of any one of claims 23 to 25.

28. A kit for detecting HSV-2 comprising a first isolated oligonucleotide of SEQ ID NO: 5 and a second oligonucleotide of any one of claims 20 to 22.

29. A kit for detecting HSV-2 comprising a first isolated oligonucleotide of SEQ ID NO: 4 and a second oligonucleotide of SEQ ID NO: 5.

30. A kit for detecting HSV-2 DNA comprising a first oligonucleotide selected from the group consisting of:
(A) an isolated oligonucleotide of the sequence SEQ ID NO: 4;
(B) an isolated oligonucleotide that hybridizes the complement of SEQ ID NO: 4 under stringent conditions and is capable of amplifying an HSV-2 glycoprotein G gene when used in conjunction with SEQ ID NO: 5 in an polymerase chain reaction; and
(C) an isolated oligonucleotide of the sequence of SEQ ID NO: 4, wherein from about one to about three nucleotides are added or removed from the 5′ end and/or from about one to about three nucleotides are added or removed from the 3′ end, respectively, and wherein the oligonucleotide is capable of amplifying an HSV-2 glycoprotein G gene when used in conjunction with SEQ ID NO: 5 in an polymerase chain reaction;
and a second oligonucleotide selected from the group consisting of
(A) an isolated oligonucleotide of the sequence SEQ ID NO: 5;
(B) an isolated oligonucleotide that hybridizes the complement of SEQ ID NO: 5 under stringent conditions and is capable of amplifying an HSV-2 glycoprotein G gene when used in conjunction with SEQ ID NO: 4 in an polymerase chain reaction; and
(C) an isolated oligonucleotide of the sequence of SEQ ID NO: 5, wherein from about one to about three nucleotides are added or removed from the 5′ end and/or from about one to about three nucleotides are added or removed from the 3′ end, respectively, and wherein the oligonucleotide is capable of amplifying an HSV-2 glycoprotein G gene when used in conjunction with SEQ ID NO: 4 in an polymerase chain reaction.

31. The kit of claims 29 or 30 further comprising SEQ ID No: 6 or a sequence wherein about one to about three nucleotides are added or removed from the 5′ end and/or about one to about three nucleotides are added or removed from the 3′ end of the sequence set forth in SEQ ID NO: 6.

32. The kit of claims 29 or 30 further comprising a PCR reaction buffer and DNA polymerase enzyme.

33. A method of detecting the presence of HSV-2 in a biological sample comprising:
(A) obtaining a biological sample from an organism;
(B) isolating nucleic acids from said sample;
(C) performing a polymerase chain reaction on said isolated nucleic acids using a first isolated oligonucleotide selected from the group consisting of:
(i) an isolated oligonucleotide of the sequence SEQ ID NO: 4;
(ii) an isolated oligonucleotide that hybridizes the complement of SEQ ID NO: 4 under stringent conditions and is capable of amplifying an HSV-2 glycoprotein G gene when used in conjunction with SEQ ID NO: 5 in an polymerase chain reaction; and
(iii) an isolated oligonucleotide of the sequence of SEQ ID NO: 4, wherein from about one to about three nucleotides are added or removed from the 5′ end and/or from about one to about three nucleotides are added or removed from the 3′ end, respectively, and wherein the oligonucleotide is capable of amplifying an HSV-2 glycoprotein G gene when used in conjunction with SEQ ID NO: 5 in an polymerase chain reaction;
and a second oligonucleotide selected from the group consisting of
(i) an isolated oligonucleotide of the sequence SEQ ID NO: 5;
(ii) an isolated oligonucleotide that hybridizes the complement of SEQ ID NO: 5 under stringent conditions and is capable of amplifying an HSV-2 glycoprotein G gene when used in conjunction with SEQ ID NO: 4 in an polymerase chain reaction; and
(iii) an isolated oligonucleotide of the sequence of SEQ ID NO: 5, wherein from about one to about three nucleotides are added or removed from the 5′ end and/or from about one to about three nucleotides are added or removed from the 3′ end, respectively, and wherein the oligonucleotide is capable of amplifying an HSV-2 glycoprotein G gene when used in conjunction with SEQ ID NO: 4 in an polymerase chain reaction,
(D) correlating a presence of an amplification product from said polymerase chain reaction with the presence of an HSV-2 in said sample.

34. The method of claim 33 wherein, the biological sample is selected from the group consisting of a tissue sample, whole blood or serum, sputum, stool, urine, semen, pericardial fluid, nasopharyngeal/throat swabs, cerebrospinal fluid (CSF), and amniotic fluid.

35. The method 33 wherein the organism is a patient suspected of having being infected by an HSV-2.

36. The method of claim 33 where the polymerase chain reaction is a real-time polymerase chain reaction.

37. The method of identifying compounds capable of inhibiting HSV-2 growth comprising:
(A) infecting a tissue culture with an HSV-2 to obtain an infected tissue culture;
(B) contacting a portion of said infected tissue culture with a compound suspected of being capable of inhibiting HSV-2 growth;
(C) isolating nucleic acids from the portion of said infected tissue culture contacted by said compound to obtain a first nucleic acid sample and from a portion of the remainder of the infected tissue culture not contacted by said compound to obtain a second nucleic acid sample;
(D) performing polymerase chain reaction on said first and said second nucleic acid samples, using a first isolated oligonucleotide selected from the group consisting of:
(i) an isolated oligonucleotide of the sequence SEQ ID NO: 4;
(ii) an isolated oligonucleotide that hybridizes the complement of SEQ ID NO: 4 under stringent conditions and is capable of amplifying an HSV-2 glycoprotein G gene when used in conjunction with SEQ ID NO: 5 in an polymerase chain reaction; and
(iii) an isolated oligonucleotide of the sequence of SEQ ID NO: 4, wherein from about one to about three nucleotides are added or removed from the 5′ end and/or from about one to about three nucleotides are added or removed from the 3′ end, respectively, and wherein the oligonucleotide is capable of amplifying an HSV-2 glycoprotein G gene when used in conjunction with SEQ ID NO: 5 in an polymerase chain reaction;
and a second oligonucleotide selected from the group consisting of
(i) an isolated oligonucleotide of the sequence SEQ ID NO: 5;
(ii) an isolated oligonucleotide that hybridizes the complement of SEQ ID NO: 5 under stringent conditions and is capable of amplifying an HSV-2 glycoprotein G gene when used in conjunction with SEQ ID NO: 4 in an polymerase chain reaction; and
(iii) an isolated oligonucleotide of the sequence of SEQ ID NO: 5, wherein from about one to about three nucleotides are added or removed from the 5′ end and/or from about one to about three nucleotides are added or removed from the 3′ end, respectively, and wherein the oligonucleotide is capable of amplifying an HSV-2 glycoprotein G gene when used in conjunction with SEQ ID NO: 4 in an polymerase chain reaction,
(D) whereby a decrease in an amplification product in the first nucleic acid sample relative to the second nucleic acid sample indicates that the compound is capable of inhibiting HSV-2 growth.

38. The method of claim 37 wherein said tissue culture comprises cells derived from the group consisting of African Green Money (Vero cells), Human HEp-2 cells, and new born mice.
